(19) United States
(12) Patent Application Publication
(10) Pub. No.: US 2019/0000753 A1
(43) Pub. Date: Jan. 3, 2019

(54) NASAL DOSAGE FORMS OF DIHYDROERGOTAMINE

(71) Applicant: Dr. Reddy's Laboratories Ltd., Hyderabad (IN)

(72) Inventors: Aditya NARASIMHA MURTHY, Hubli (IN); Piyush GUPTA, Ghaziabad (IN); Arun JANA, West Midnapur (IN); Vishal VALLABHADAS RATHI, Miyapur Hyderabad (IN); Girish KARANTH, Miyapur Hyderabad (IN); Rajeev Singh RAGHUVANSHI, South City - I, Gurgaon (IN)

(21) Appl. No.: 16/025,629

(22) Filed: Jul. 2, 2018

(30) Foreign Application Priority Data
Jul. 2, 2017 (IN) 201741000065

(51) Int. Cl. A61K 9/00; A61K 31/4985; A61K 47/26; A61K 47/10; A61K 47/02; A61K 47/20; A61K 47/12; A61K 47/18; A61K 47/34; A61K 47/32

(52) U.S. Cl. CPC A61K 9/0043; A61K 31/4985; A61K 47/26; A61K 47/10; A61K 47/02; A61K 47/186; A61K 47/12; A61K 47/183; A61K 47/34; A61K 47/32; A61K 47/20

(57) ABSTRACT

The present application relates to a nasal dosage form of dihydroergotamine, wherein said dosage form requires less than about 15 minutes for administration and requires less than four sprays to administer effective dose of dihydroergotamine for treating migraine in human subjects.

### FIELD OF THE INVENTION

The present application relates to a nasal dosage form of dihydroergotamine, wherein said dosage form requires less than about 15 minutes for administration and requires less than four sprays to administer effective dose of dihydroergotamine for treating migraine in human subjects.

### BACKGROUND

Migraine is a type of headache, which is a severe, seriously debilitating and usually unilateral form of episodic headache that may be preceded by aura and that is frequently associated with both neurological and gastrointestinal symptoms such as nausea, vomiting, diarrhea, sensitivity to light (photophobia), sound (phonophobia), and smells (osmophobia); sleep disruption, and depression. When untreated, a migraine headache attack may last anywhere from 4 to 72 hours.

Dihydroergotamine mesylate (DHE) has been used in migraine therapy for a long time. In patients with migraine attacks DHE is administered through parenteral route. Dihydroergotamine has been marketed as a nasal spray, alternative to the parenteral route of administration. The nasal spray seems to be a good alternative, because it is painless, less expensive and less inconvenient than injection. However, nausea and vomiting are commonly seen in migraine patients, making them to choose nasal spray than oral treatment.

Nasal dosage form of dihydroergotamine is approved in U.S under the brand name MIGRANAL® (NDA no. 020148) and used in acute treatment of migraine with or without aura in adults.

Administration of MIGRANAL® nasal spray is a tedious and time consuming process. The administration process comprises following steps—First, the patient should remove the metal seal and stopper from the vial and fix the spray pump (the vial should be discarded within 8 hours of opening); Second, the vial should be pumped (primed) 4 times away before the actual use (care should be taken, not to pump more than 4 times); Third, it should be sprayed into each nostril (0.5 mg each nostril), without tilting the head; and Fourth, wait for 15 minutes and spray once again into each nostril, to complete the administration of 2.0 mg. This current administration method for Migranal® takes minimum of 20 minutes to complete the process. This is definitely cumbersome for patients, especially during migraine attacks.

Currently there are no nasal dosage forms available for dihydroergotamine that offers rapid and easy administration, to provide effective dose.

There remains a long felt need to develop a pharmaceutical nasal dosage form of dihydroergotamine for rapid and easy administration of effective dose, for treating migraine with or without aura in human subjects.

There is also a need for a pharmaceutical nasal dosage form of dihydroergotamine which does not require any priming before use or ready to use nasal device. In other words there is a need for a pre-primed pharmaceutical nasal dosage form of dihydroergotamine, for treating migraine with or without aura in human subjects.

It is desired to have a pharmaceutical nasal dosage form of dihydroergotamine which requires less than about 15 minutes for administration and minimizes the number of sprays, to administer effective dose of dihydroergotamine for treating migraine with or without aura in human subjects.

The present application relates to a pharmaceutical nasal dosage form of dihydroergotamine which requires less than about 15 minutes for administration and minimizes the number of sprays to less than four sprays, to administer effective dose of dihydroergotamine for treating migraine with or without aura in human subjects.

### DEFINITIONS

Unless otherwise defined, all technical and scientific terms used herein have the same meaning as commonly understood by one of ordinary skill in the art.

The terms “about,” “up to,” “generally,” and the like are to be construed as modifying a term or value such that it is not an absolute. Such terms will be defined by the circumstances and the terms that they modify as those terms are understood by those of skill in the art. This includes, at very least, the degree of expected experimental error, technical error and instrumental error for a given experiment, technique or an instrument used to measure a value. The term “about” is used to provide flexibility to a numerical range endpoint by providing that a given value may be “a little above” or “a little below” the endpoint. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum.

The term “dihydroergotamine” as used herein refers to dihydroergotamine or a pharmaceutically acceptable salt(s) such as dihydroergotamine mesylate, dihydroergotamine tartrate and the like.

The term “commercially available dihydroergotamine nasal spray” as used herein refers to MIGRANAL® nasal spray available in 3.5 mL amber glass vials containing 4 mg of dihydroergotamine mesylate, USP (NDA no. 020148) marketed by Valeant Pharmaceuticals Inc. or its pharmaceutical equivalents or its therapeutic equivalents or later approved drugs which are designated as AB rated by US FDA as per Approved Drug Products with Therapeutic Equivalence Evaluations (34th edition or any later published editions) or drugs that have obtained marketing approval by US FDA through Abbreviated New Drug Application (ANDA) filing by establishing bioequivalence to such Product.

The term “device” or “nasal device,” or “nasal spray device,” as used herein, refers to any apparatus that is capable of delivering/spraying the effective dose of dihydroergotamine or a pharmaceutically acceptable salt thereof, into the nostril/nasal cavity of a patient in a need thereof.

The term “pre-primed,” as used herein, refers to a device, such as a nasal spray device which is capable of delivering the nasal dosage form of dihydroergotamine and at least one pharmaceutically acceptable excipient to a patient in need thereof, with the first actuation of the spray pump, i.e., without the need to prime (pumping the nasal spray) the pump prior to dosing, such as by actuating/pushing the pump one or more times until the spray appears.

The term “migraine” as used herein refers to migraine with or without aura.

The term “treating migraine” as used herein refers to treatment of acute migraine attacks with or without aura.

The term “treating cluster headache” as used herein refers to treatment of cluster headache episodes.

The term “human subject” as used herein refers to a human may or may not be suffering from migraine or cluster headache.

The term “dC/dT” as used herein, refers to change in dihydroergotamine concentration in plasma as a function of time or change in plasma concentration of dihydroergotamine during said time period or interval. It is calculated as

$$dC / dT = \frac{( Plasma {Concentration}_{T 2} - Plasma {Concentration}_{T 1} )}{( Time {point}_{2} - Time {point}_{1} )}$$

### DESCRIPTION OF THE EMBODIMENTS

The present application relates to a pharmaceutical nasal dosage form of dihydroergotamine, wherein said dosage form requires less than about 15 minutes for administration and minimizes the number of sprays to less than four sprays to administer effective dose of dihydroergotamine for treating migraine with or without aura or cluster headache in human subjects.

In one embodiment, the present application relates to a pharmaceutical nasal dosage form of dihydroergotamine for treating migraine with or without aura in human subjects, wherein said dosage form requires less than about 15 minutes for administration and minimizes the number of sprays to less than four sprays to administer effective dose of dihydroergotamine.

In another embodiment, the present application relates to a pharmaceutical nasal dosage form of dihydroergotamine for treating migraine with or without aura in human subjects, wherein said dosage form requires less than about 15 minutes for administration and minimizes the number of sprays to less than three sprays to administer effective dose of dihydroergotamine.

In another embodiment, the present application relates to a pharmaceutical nasal dosage form of dihydroergotamine for treating migraine with or without aura in human subjects, wherein said dosage form requires less than about 15 minutes for administration and minimizes the number of sprays to less than two sprays to administer effective dose of dihydroergotamine.

In another embodiment, the present application relates to a pharmaceutical nasal dosage form of dihydroergotamine for treating migraine with or without aura in human subjects, wherein said dosage form requires less than about 15 minutes for administration and requires not more than two sprays to administer effective dose of dihydroergotamine.

In another embodiment, the present application relates to a pharmaceutical nasal dosage form of dihydroergotamine for treating migraine with or without aura in human subjects, wherein said dosage form requires less than about 15 minutes for administration and requires at least one spray to administer effective dose of dihydroergotamine.

In an aspect of the above embodiments, the pharmaceutical nasal dosage form of the present application can be provided in the form of aqueous solution, suspension, emulsion, aerosol, powder and the like.

In another aspect of the above embodiments, the pharmaceutical nasal dosage form of the present application can be provided in a suitable pre-primed nasal device such as mono dose or bi-dose device for administering effective dose of dihydroergotamine for treating migraine with or without aura in human subjects.

In another aspect of the above embodiments, the present application relates to a pharmaceutical nasal dosage form comprising aqueous solution of dihydroergotamine or a pharmaceutically acceptable salt thereof, for treating migraine with or without aura in human subjects, wherein said dosage form is provided in a pre-primed nasal device and said dosage form requires less than about 15 minutes for administration and minimizes the number of sprays to less than four sprays to administer effective dose of dihydroergotamine.

In another aspect of the above embodiments, the present application relates to a pharmaceutical nasal dosage form comprising aqueous solution of dihydroergotamine or a pharmaceutically acceptable salt thereof, for treating migraine with or without aura in human subjects, wherein said dosage form is provided in a pre-primed nasal device and said dosage form requires less than about 15 minutes for administration and minimizes the number of sprays to less than three sprays to administer effective dose of dihydroergotamine.

In another aspect of the above embodiments, the present application relates to a pharmaceutical nasal dosage form comprising aqueous solution of dihydroergotamine or a pharmaceutically acceptable salt thereof, for treating migraine with or without aura in human subjects, wherein said dosage form is provided in a pre-primed nasal device and said dosage form requires less than about 15 minutes for administration and minimizes the number of sprays to less than two sprays to administer effective dose of dihydroergotamine.

In another aspect of the above embodiments, the present application relates to a pharmaceutical nasal dosage form comprising aqueous solution of dihydroergotamine or a pharmaceutically acceptable salt thereof, for treating migraine with or without aura in human subjects, wherein said dosage form is provided in a pre-primed nasal device and said dosage form requires less than about 15 minutes for administration and requires not more than two sprays to administer effective dose of dihydroergotamine.

In another aspect of the above embodiments, the present application relates to a pharmaceutical nasal dosage form comprising aqueous solution of dihydroergotamine or a pharmaceutically acceptable salt thereof, for treating migraine with or without aura in human subjects, wherein said dosage form is provided in a pre-primed nasal device and said dosage form requires less than about 15 minutes for administration and requires at least one spray to administer effective dose of dihydroergotamine.

In another aspect of the above embodiments, the pharmaceutical nasal dosage form of the present application comprises dihydroergotamine from about 0.5 mg to about 2.0 mg.

In another aspect of the above embodiments, the present application relates to a pharmaceutical nasal dosage form of dihydroergotamine for treating migraine with or without aura in human subjects, wherein said dosage form comprises dihydroergotamine from about 0.5 mg to about 2.0 mg and at least one pharmaceutically acceptable excipient.

In another aspect of the above embodiments, the present application relates to a pharmaceutical nasal dosage form of dihydroergotamine for treating migraine with or without aura in human subjects, wherein said dosage form comprises less than about 2 mg of dihydroergotamine and at least one pharmaceutically acceptable excipient.

In another aspect of the above embodiments, the present application relates to a pharmaceutical nasal dosage form of dihydroergotamine for treating migraine with or without aura in human subjects, wherein said dosage form comprises at least about 1.6 mg of dihydroergotamine and at least one pharmaceutically acceptable excipient.

In another aspect of the above embodiments, the present application relates to a pharmaceutical nasal dosage form of dihydroergotamine for treating migraine with or without aura in human subjects, wherein said dosage form comprises at least about 1.3 mg of dihydroergotamine and at least one pharmaceutically acceptable excipient.

In another aspect of the above embodiments, the present application relates to a pharmaceutical nasal dosage form of dihydroergotamine for treating migraine with or without aura in human subjects, wherein said dosage form comprises at least about 1.2 mg of dihydroergotamine and at least one pharmaceutically acceptable excipient.

In another aspect of the above embodiments, the present application relates to a pharmaceutical nasal dosage form of dihydroergotamine for treating migraine with or without aura in human subjects, wherein said dosage form comprises at least about 1.1 mg of dihydroergotamine and at least one pharmaceutically acceptable excipient.

In another aspect of the above embodiments, the pharmaceutical nasal dosage form of dihydroergotamine requires less than about 15 minutes for administration and minimizes the number of sprays to less than four sprays to administer effective dose of dihydroergotamine, wherein said effective dose is less than about 2 mg of dihydroergotamine.

In another aspect of the above embodiments, the pharmaceutical nasal dosage form of dihydroergotamine requires less than about 15 minutes for administration and minimizes the number of sprays to less than three sprays to administer effective dose of dihydroergotamine, wherein said effective dose is less than about 2 mg of dihydroergotamine.

In another aspect of the above embodiments, the pharmaceutical nasal dosage form of dihydroergotamine requires less than about 15 minutes for administration and minimizes the number of sprays to less than two sprays to administer effective dose of dihydroergotamine, wherein said effective dose is less than about 2 mg of dihydroergotamine.

In another aspect of the above embodiments, the pharmaceutical nasal dosage form of dihydroergotamine requires less than about 15 minutes for administration and requires not more than two sprays to administer effective dose of dihydroergotamine, wherein said effective dose is less than about 2 mg of dihydroergotamine.

In another aspect of the above embodiments, the pharmaceutical nasal dosage form of dihydroergotamine requires less than about 15 minutes for administration and requires at least one spray to administer effective dose of dihydroergotamine, wherein said effective dose is less than about 2 mg of dihydroergotamine.

In another aspect of the above embodiments, the pharmaceutical nasal dosage form of dihydroergotamine requires less than about 15 minutes for administration and minimizes the number of sprays to less than four sprays to administer effective dose of dihydroergotamine, wherein said effective dose is about 1.6 mg of dihydroergotamine.

In another aspect of the above embodiments, the pharmaceutical nasal dosage form of dihydroergotamine requires less than about 15 minutes for administration and minimizes the number of sprays to less than three sprays to administer effective dose of dihydroergotamine, wherein said effective dose is about 1.6 mg of dihydroergotamine.

In another aspect of the above embodiments, the pharmaceutical nasal dosage form of dihydroergotamine requires less than about 15 minutes for administration and minimizes the number of sprays to less than two sprays to administer effective dose of dihydroergotamine, wherein said effective dose is about 1.6 mg of dihydroergotamine.

In another aspect of the above embodiments, the pharmaceutical nasal dosage form of dihydroergotamine requires less than about 15 minutes for administration and requires not more than two sprays to administer effective dose of dihydroergotamine, wherein said effective dose is about 1.6 mg of dihydroergotamine.

In another aspect of the above embodiments, the pharmaceutical nasal dosage form of dihydroergotamine requires less than about 15 minutes for administration and requires at least one spray to administer effective dose of dihydroergotamine, wherein said effective dose is about 1.6 mg of dihydroergotamine.

In another aspect of the above embodiments, the pharmaceutical nasal dosage form of dihydroergotamine requires less than about 15 minutes for administration and minimizes the number of sprays to less than four sprays to administer effective dose of dihydroergotamine, wherein said effective dose is about 1.3 mg of dihydroergotamine.

In another aspect of the above embodiments, the pharmaceutical nasal dosage form of dihydroergotamine requires less than about 15 minutes for administration and minimizes the number of sprays to less than three sprays to administer effective dose of dihydroergotamine, wherein said effective dose is about 1.3 mg of dihydroergotamine.

In another aspect of the above embodiments, the pharmaceutical nasal dosage form of dihydroergotamine requires less than about 15 minutes for administration and minimizes the number of sprays to less than two sprays to administer effective dose of dihydroergotamine, wherein said effective dose is about 1.3 mg of dihydroergotamine.

In another aspect of the above embodiments, the pharmaceutical nasal dosage form of dihydroergotamine requires less than about 15 minutes for administration and requires not more than two sprays to administer effective dose of dihydroergotamine, wherein said effective dose is about 1.3 mg of dihydroergotamine.

In another aspect of the above embodiments, the pharmaceutical nasal dosage form of dihydroergotamine requires less than about 15 minutes for administration and requires at least one spray to administer effective dose of dihydroergotamine, wherein said effective dose is about 1.3 mg of dihydroergotamine.

In an aspect of the above embodiments, the pharmaceutical nasal dosage form of dihydroergotamine of the present application can be provided in the form of aqueous solution, suspension, emulsion, aerosol, powder and the like.

In another aspect of the above embodiments, the pharmaceutical nasal dosage form of dihydroergotamine requires less than about 15 minutes for administration and minimizes the number of sprays to less than four sprays to administer effective dose of dihydroergotamine, wherein said effective dose is about 1.2 mg of dihydroergotamine.

In another aspect of the above embodiments, the pharmaceutical nasal dosage form of dihydroergotamine requires less than about 15 minutes for administration and minimizes the number of sprays to less than three sprays to administer effective dose of dihydroergotamine, wherein said effective dose is about 1.2 mg of dihydroergotamine.

In another aspect of the above embodiments, the pharmaceutical nasal dosage form of dihydroergotamine requires less than about 15 minutes for administration and minimizes the number of sprays to less than two sprays to administer effective dose of dihydroergotamine, wherein said effective dose is about 1.2 mg of dihydroergotamine.

In another aspect of the above embodiments, the pharmaceutical nasal dosage form of dihydroergotamine requires less than about 15 minutes for administration and requires not more than two sprays to administer effective dose of dihydroergotamine, wherein said effective dose is about 1.2 mg of dihydroergotamine.

In another aspect of the above embodiments, the pharmaceutical nasal dosage form of dihydroergotamine requires less than about 15 minutes for administration and requires at least one spray to administer effective dose of dihydroergotamine, wherein said effective dose is about 1.2 mg of dihydroergotamine.

In an aspect of the above embodiments, the pharmaceutical nasal dosage form of dihydroergotamine of the present application can be provided in the form of aqueous solution, suspension, emulsion, aerosol, powder and the like.

In another aspect of the above embodiments, the pharmaceutical nasal dosage form of dihydroergotamine requires less than about 15 minutes for administration and minimizes the number of sprays to less than four sprays to administer effective dose of dihydroergotamine, wherein said effective dose is about 1.1 mg of dihydroergotamine.

In another aspect of the above embodiments, the pharmaceutical nasal dosage form of dihydroergotamine requires less than about 15 minutes for administration and minimizes the number of sprays to less than three sprays to administer effective dose of dihydroergotamine, wherein said effective dose is about 1.1 mg of dihydroergotamine.

In another aspect of the above embodiments, the pharmaceutical nasal dosage form of dihydroergotamine requires less than about 15 minutes for administration and minimizes the number of sprays to less than two sprays to administer effective dose of dihydroergotamine, wherein said effective dose is about 1.1 mg of dihydroergotamine.

In another aspect of the above embodiments, the pharmaceutical nasal dosage form of dihydroergotamine requires less than about 15 minutes for administration and requires not more than two sprays to administer effective dose of dihydroergotamine, wherein said effective dose is about 1.1 mg of dihydroergotamine.

In another aspect of the above embodiments, the pharmaceutical nasal dosage form of dihydroergotamine requires less than about 15 minutes for administration and requires at least one spray to administer effective dose of dihydroergotamine, wherein said effective dose is about 1.1 mg of dihydroergotamine.

In aspect of the above embodiments, the pharmaceutical nasal dosage form of dihydroergotamine of the present application can be provided in the form of aqueous solution, suspension, emulsion, aerosol, powder and the like.

In aspect of the above embodiments, the present application relates to a pharmaceutical nasal dosage form comprising aqueous solution of dihydroergotamine or a pharmaceutically acceptable salt thereof, at a concentration from about 0.5 mg/0.1 ml to about 2 mg/0.1 ml for treating migraine with or without aura in human subjects, wherein said dosage form is provided in a pre-primed nasal device and said dosage form requires less than about 15 minutes for administration and minimizes the number of sprays to less than four sprays to administer effective dose of dihydroergotamine.

In another aspect of the above embodiments, the present application relates to a pharmaceutical nasal dosage form of dihydroergotamine for treating migraine with or without aura in human subjects, wherein said dosage form comprises dihydroergotamine from about 0.5 mg/0.1 ml to about 2.0 mg/0.1 ml and at least one pharmaceutically acceptable excipient.

In another aspect of the above embodiments, the pharmaceutical nasal dosage form of dihydroergotamine requires less than about 15 minutes for administration and requires at least one spray to administer effective dose of dihydroergotamine, wherein said effective dose is from about to 0.5 mg/0.1 ml to about 2 mg/0.1 ml of dihydroergotamine.

In another aspect of the above embodiments, the pharmaceutical nasal dosage form of dihydroergotamine requires less than about 15 minutes for administration and minimizes the number of sprays to less than four sprays to administer effective dose of dihydroergotamine, wherein said effective dose is about 0.8 mg/0.1 ml of dihydroergotamine.

In another aspect of the above embodiments, the pharmaceutical nasal dosage form of dihydroergotamine requires less than about 15 minutes for administration and minimizes the number of sprays to less than four sprays to administer effective dose of dihydroergotamine, wherein said effective dose is about 0.65 mg/0.1 ml of dihydroergotamine.

In another aspect of the above embodiments, the pharmaceutical nasal dosage form of dihydroergotamine requires less than about 15 minutes for administration and minimizes the number of sprays to less than four sprays to administer effective dose of dihydroergotamine, wherein said effective dose is about 0.6 mg/0.1 ml of dihydroergotamine.

In another aspect of the above embodiments, the pharmaceutical nasal dosage form of dihydroergotamine requires less than about 15 minutes for administration and minimizes the number of sprays to less than four sprays to administer effective dose of dihydroergotamine, wherein said effective dose is about 0.55 mg/0.1 ml of dihydroergotamine.

In another aspect of the above embodiments, the present application relates to a pharmaceutical nasal dosage form of dihydroergotamine for treating migraine with or without aura in human subjects, wherein said dosage form comprises aqueous solution of dihydroergotamine from about 0.5 mg/0.1 ml to about 2.0 mg/0.1 ml and at least one pharmaceutically acceptable excipient.

In another aspect of the above embodiments, the pharmaceutical nasal dosage form of dihydroergotamine of present application further comprises one or more stabilizers.

Suitable stabilizers used in the present application includes, but are not limited to, amino acids such as lysine phenylalanine, leucine and the like, sugars including raffinose, inulin and the like, butylated hydroxyanisole, butylated hydroxytoluene, monothioglycerol, propyl gallate, sodium ascorbate, sodium bisulfite, sodium metabisulfite, sodium citrate, potassium metabisulfite, potassium sulfite, ammonium acetate, sodium sulfite, chelating agent includes, but are not limited to EDTA, polycarboxylic acids or acids and salts thereof such as citric acid, tartaric acid, ascorbic acid, dehydroascorbic acid, acetic acid, formic acid, methanoic acid, fumaric acid, propionic acid, butanoic acid, ethanoic acid, benzoic acid, butyric acid, malic acid, propionic acid, epoxysuccinic acid, muconic acid, furanacrylic acid, citramalic acid, capric acid, stearic acid, caproic acid, malonic acid, succinic acid, diethylacetic acid, methylbutyric acid, and the like and combinations thereof, other acids such as hydrochloric acid, hydrobromic acid, phosphoric acid, nitric acid, and sulfuric acid, Surfactants such as nonionic, anionic, cationic or zwitterionic surfactants. Nonionic surfactants include, but are not limited to, Pluronic®, Tweens®, Spans®, Polysorbate® 80, Polyoxyethylene sorbitan oleate; Polyethylene oxide sorbitan mono-oleate; Polyoxyethylene (20) sorbitan monooleate, vitamin E derivatives such as tocopherol succinate (TOS), D-α-tocopheryl polyethylene glycol succinate (Vitamin E TPGS or TPGS), D-α-tocopheryl polyethylene glycol 1000 succinate (Vitamin E TPGSi000) and D-α-tocopherol polyethylene glycol 2000 succinate (TPGS₂₀₀₀) and the like and combinations thereof.

In another aspect of the above embodiments, the pharmaceutical nasal dosage form of dihydroergotamine of the present application comprises one or more stabilizers in an amount of from about 0.01% w/w to about 10% w/w, or from about 0.01% w/w to about 5% w/w, or from about 0.01% w/w to about 2% w/w, or from about 0.05% w/w to about 2% w/w, or from about 0.01% w/w to about 1.5% w/w, or from about 0.1% w/w to about 1% w/w, based on the total weight of the composition/dosage form.

In one aspect of the above embodiments, the pharmaceutical nasal dosage form of dihydroergotamine of the present application comprises dihydroergotamine or a pharmaceutically acceptable salt thereof, sodium citrate, citric acid or combinations thereof for treating migraine with or without aura in human subjects, wherein said dosage form is administered using a pre-primed nasal device and said dosage form requires less than about 15 minutes to administer effective dose of dihydroergotamine.

In one aspect of the above embodiments, the pharmaceutical nasal dosage form of dihydroergotamine of the present application comprises dihydroergotamine or a pharmaceutically acceptable salt thereof, ammonium acetate, acetic acid or combinations thereof for treating migraine with or without aura in human subjects, wherein said dosage form is administered using a pre-primed nasal device and said dosage form requires less than about 15 minutes to administer effective dose of dihydroergotamine.

In one aspect of the above embodiments, the pharmaceutical nasal dosage form of dihydroergotamine of the present application comprises dihydroergotamine or a pharmaceutically acceptable salt thereof, and ascorbic acid for treating migraine with or without aura in human subjects, wherein said dosage form is administered using a pre-primed nasal device and said dosage form requires less than about 15 minutes to administer effective dose of dihydroergotamine.

In one aspect of the above embodiments, the pharmaceutical nasal dosage form of dihydroergotamine of the present application comprises dihydroergotamine or a pharmaceutically acceptable salt thereof, and vitamin E TPGS for treating migraine with or without aura in human subjects, wherein said dosage form is administered using a pre-primed nasal device and said dosage form requires less than about 15 minutes to administer effective dose of dihydroergotamine.

In another aspect of the above embodiments, the pharmaceutical nasal dosage form of dihydroergotamine comprises dihydroergotamine and stabilizers, wherein said dosage form does not show any precipitation upon storage for at least 7 days, at least 10 days, at least 11 days, at least 12 days, at least 13 days, at least 20 days, at least 30 days, at least 45 days, at least 60 days or longer.

In another aspect of the above embodiments, the pharmaceutical nasal dosage form of dihydroergotamine comprises dihydroergotamine and stabilizers, wherein said dosage form does not show any precipitation upon storage such as at 2° C. to 8° C., 25° C., 40° C., or 45° C. for at least 7 days, at least 10 days, at least 11 days, at least 12 days, at least 13 days, at least 20 days, at least 30 days, at least 45 days, at least 60 days or longer.

In another aspect of the above embodiments, the pharmaceutical nasal dosage form of dihydroergotamine comprises dihydroergotamine and stabilizers, wherein said dosage form does not show any precipitation upon storage at 40° C. for at least 7 days, at least 10 days, at least 11 days, at least 12 days, at least 13 days, at least 20 days, at least 30 days, at least 45 days, at least 60 days or longer.

In another aspect of the above embodiments, the pharmaceutical nasal dosage form of dihydroergotamine comprises dihydroergotamine and stabilizers, wherein said dosage form does not show any precipitation upon storage at 45° C. for at least 7 days, at least 10 days, at least 11 days, at least 12 days, at least 13 days, at least 20 days, at least 30 days, at least 45 days, at least 60 days or longer.

In another aspect of the above embodiments, the pharmaceutical nasal dosage form of dihydroergotamine comprises dihydroergotamine and stabilizers in a weight ratio of from about 1.0:30.0 to about 30.0:1.0.

In another aspect of the above embodiments, the pharmaceutical nasal dosage form of dihydroergotamine comprises dihydroergotamine and stabilizers selected from the group of sodium citrate, citric acid or combinations thereof, wherein said dosage form does not show any precipitation upon storage such as at 2° C. to 8° C., 25° C., 40° C., or 45° C. for at least 7 days, at least 10 days, at least 11 days, at least 12 days, at least 13 days, at least 20 days, at least 30 days, at least 45 days, at least 60 days or longer.

In another aspect of the above embodiments, the pharmaceutical nasal dosage form of dihydroergotamine comprises dihydroergotamine and stabilizers selected from the group of ammonium acetate, acetic acid or combinations thereof, wherein said dosage form does not show any precipitation upon storage such as at 2° C. to 8° C., 25° C., 40° C., or 45° C. for at least 7 days, at least 10 days, at least 11 days, at least 12 days, at least 13 days, at least 20 days, at least 30 days, at least 45 days, at least 60 days or longer.

In another aspect of the above embodiments, the pharmaceutical nasal dosage form of dihydroergotamine comprises dihydroergotamine and ascorbic acid, wherein said dosage form does not show any precipitation upon storage such as at 2° C. to 8° C., 25° C., 40° C., or 45° C. for at least 7 days, at least 10 days, at least 11 days, at least 12 days, at least 13 days, at least 20 days, at least 30 days, at least 45 days, at least 60 days or longer.

In another aspect of the above embodiments, the pharmaceutical nasal dosage form of dihydroergotamine comprises dihydroergotamine and stabilizers selected from the group of sodium citrate, citric acid or combinations thereof, wherein said dosage form does not show any precipitation upon storage at 45° C. for at least 7 days, at least 10 days, at least 11 days, at least 12 days, at least 13 days, at least 20 days, at least 30 days, at least 45 days, at least 60 days or longer.

In another aspect of the above embodiments, the pharmaceutical nasal dosage form of dihydroergotamine comprises dihydroergotamine, sodium citrate and citric acid or combinations thereof, wherein said dosage form does not show any precipitation upon storage such as at 2° C. to 8° C., 25° C., 40° C., or 45° C. for at least 7 days, at least 10 days, at least 11 days, at least 12 days, at least 13 days, at least 20 days, at least 30 days, at least 45 days, at least 60 days or longer.

In another aspect of the above embodiments, the pharmaceutical nasal dosage form of dihydroergotamine comprises dihydroergotamine, sodium citrate and citric acid or combinations thereof, wherein said dosage form does not show any precipitation upon storage at 45° C. for at least 7 days, at least 10 days, at least 11 days, at least 12 days, at least 13 days, at least 20 days, at least 30 days, at least 45 days, at least 60 days or longer.

In another aspect of the above embodiments, the pharmaceutical nasal dosage form of dihydroergotamine comprises dihydroergotamine and stabilizers selected from the group of ammonium acetate, acetic acid or combinations thereof, wherein said dosage form does not show any precipitation upon storage at 45° C. for at least 7 days, at least 10 days, at least 11 days, at least 12 days, at least 13 days, at least 20 days, at least 30 days, at least 45 days, at least 60 days or longer.

In another aspect of the above embodiments, the pharmaceutical nasal dosage form of dihydroergotamine comprises dihydroergotamine, ammonium acetate and acetic acid or combinations thereof, wherein said dosage form does not show any precipitation upon storage such as at 2° C. to 8° C., 25° C., 40° C., or 45° C. for at least 7 days, at least 10 days, at least 11 days, at least 12 days, at least 13 days, at least 20 days, at least 30 days, at least 45 days, at least 60 days or longer.

In another aspect of the above embodiments, the pharmaceutical nasal dosage form of dihydroergotamine comprises dihydroergotamine, ammonium acetate and acetic acid or combinations thereof, wherein said dosage form does not show any precipitation upon storage at 45° C. for at least 7 days, at least 10 days, at least 11 days, at least 12 days, at least 13 days, at least 20 days, at least 30 days, at least 45 days, at least 60 days or longer.

In another aspect of the above embodiments, the pharmaceutical nasal dosage form of dihydroergotamine comprises dihydroergotamine and acid, wherein said dosage form does not show any precipitation upon storage such as at 2° C. to 8° C., 25° C., 40° C., or 45° C. for at least 7 days, at least 10 days, at least 11 days, at least 12 days, at least 13 days, at least 20 days, at least 30 days, at least 45 days, at least 60 days or longer.

In another aspect of the above embodiments, the pharmaceutical nasal dosage form of dihydroergotamine comprises dihydroergotamine and ascorbic acid, wherein said dosage form does not show any precipitation upon storage such as at 2° C. to 8° C., 25° C., 40° C., or 45° C. for at least 7 days, at least 10 days, at least 11 days, at least 12 days, at least 13 days, at least 20 days, at least 30 days, at least 45 days, at least 60 days or longer.

In another aspect of the above embodiments, the pharmaceutical nasal dosage form of dihydroergotamine comprises dihydroergotamine and stabilizers selected from the group of vitamin E derivatives such as vitamin E TPGS, benzalkonium chloride, or combinations thereof, wherein said dosage form does not show any precipitation upon storage such as at 2° C. to 8° C., 25° C., 40° C., or 45° C. for at least 7 days, at least 10 days, at least 11 days, at least 12 days, at least 13 days, at least 20 days, at least 30 days, at least 45 days, at least 60 days or longer.

In another aspect of the above embodiments, the pharmaceutical nasal dosage form of dihydroergotamine comprises dihydroergotamine and benzalkonium chloride, wherein said dosage form does not show any precipitation upon storage such as at 2° C. to 8° C., 25° C., 40° C., or 45° C. at least 7 days, at least 10 days, at least 11 days, at least 12 days, at least 13 days, at least 20 days, at least 30 days, at least 45 days, at least 60 days or longer.

In another aspect of the above embodiments, the pharmaceutical nasal dosage form of dihydroergotamine comprises dihydroergotamine and at least one stabilizer selected from vitamin E derivatives, wherein said dosage form does not show any precipitation upon storage such as at 2° C. to 8° C., 25° C., 40° C., or 45° C. for at least 7 days, at least 10 days, at least 11 days, at least 12 days, at least 13 days, at least 20 days, at least 30 days, at least 45 days, at least 60 days or longer.

In another aspect of the above embodiments, the pharmaceutical nasal dosage form of dihydroergotamine comprises dihydroergotamine and stabilizers, wherein said dosage form contains total impurities of not more than about 5% or 4% or 3% or 2% or 1% when evaluated for at least about 3 months at about 2° C. to about 8° C., or about 25° C. with at least about 60% relative humidity and or about 40° C. with least about 75% relative humidity.

In another aspect of the above embodiments, the pharmaceutical nasal dosage form of dihydroergotamine comprises dihydroergotamine and stabilizers, wherein said dosage form contains total impurities not more than about 5% or 4% or 3% or 2% or 1% when evaluated for at least about 6 months at about 2° C. to about 8° C., or about 25° C. with at least about 60% relative humidity and or about 40° C. with at least about 75% relative humidity.

In another aspect of the above embodiments, the pharmaceutical nasal dosage form of dihydroergotamine comprises dihydroergotamine and stabilizers selected from the group of sodium citrate, citric acid or combinations thereof, wherein said dosage form contains total impurities not more than about 5% or 4% or 3% or 2% or 1% when evaluated for at least about 6 months at about 2° C. to about 8° C.

In another aspect of the above embodiments, the pharmaceutical nasal dosage form of dihydroergotamine comprises dihydroergotamine and stabilizers selected from the group of sodium citrate, citric acid or combinations thereof, wherein said dosage form contains total impurities not more than about 5% or 4% or 3% or 2% or 1% when evaluated for at least about 6 months at about 25° C. with at least about 60% relative humidity.

In another aspect of the above embodiments, the pharmaceutical nasal dosage form of dihydroergotamine comprises dihydroergotamine and stabilizers selected from the group of sodium citrate, citric acid or combinations thereof, wherein said dosage form contains total impurities not more than about 5% or 4% or 3% or 2% or 1% when evaluated for at least about 6 months at about 40° C. with at least about 75% relative humidity.

In another aspect of the above embodiments, the pharmaceutical nasal dosage form of dihydroergotamine comprises dihydroergotamine and stabilizers selected from the group of ammonium acetate, acetic acid or combinations thereof, wherein said dosage form contains total impurities not more than about 5% or 4% or 3% or 2% or 1% when evaluated for at least about 6 months at about 2° C. to about 8° C.

In another aspect of the above embodiments, the pharmaceutical nasal dosage form of dihydroergotamine comprises dihydroergotamine and stabilizers selected from the group of ammonium acetate, acetic acid or combinations thereof, wherein said dosage form contains total impurities not more than about 5% or 4% or 3% or 2% or 1% when evaluated for at least about 6 months at about 25° C. with at least about 60% relative humidity.

In another aspect of the above embodiments, the pharmaceutical nasal dosage form of dihydroergotamine comprises dihydroergotamine and stabilizers selected from the group of ammonium acetate, acetic acid or combinations thereof, wherein said dosage form contains total impurities not more than about 5% or 4% or 3% or 2% or 1% when evaluated for at least about 6 months at about 40° C. with at least about 75% relative humidity.

In another aspect of the above embodiments, the pharmaceutical nasal dosage form of dihydroergotamine comprises dihydroergotamine and ascorbic acid, wherein said dosage form contains total impurities not more than about 5% or 4% or 3% or 2% or 1% when evaluated for at least about 6 months at about 2° C. to 8° C.

In another aspect of the above embodiments, the pharmaceutical nasal dosage form of dihydroergotamine comprises dihydroergotamine and ascorbic acid, wherein said dosage form contains total impurities not more than about 5% or 4% or 3% or 2% or 1% when evaluated for at least about 6 months at about 25° C. with at least about 60% relative humidity.

In another aspect of the above embodiments, the pharmaceutical nasal dosage form of dihydroergotamine comprises dihydroergotamine and ascorbic acid, wherein said dosage form contains total impurities not more than about 5% or 4% or 3% or 2% or 1% when evaluated for at least about 6 months at about 40° C. with at least about 75% relative humidity.

In another aspect of the above embodiments, the pharmaceutical nasal dosage form of dihydroergotamine comprises dihydroergotamine and at least one stabilizer selected from the group of vitamin E derivatives, wherein said dosage form contains total impurities not more than about 5% or 4% or 3% or 2% or 1% when evaluated for at least about 6 months at about 2° C. to 8° C., or 25° C. or 40° C., with at least about 30% relative humidity to about 75% relative humidity.

In another aspect of the above embodiments, the pharmaceutical nasal dosage form of dihydroergotamine comprises dihydroergotamine and at least one stabilizer selected from the group of vitamin E derivatives, wherein said dosage form contains total impurities not more than about 5% or 4% or 3% or 2% or 1% when evaluated for at least about 6 months at about 2° C. to 8° C.

In another aspect of the above embodiments, the pharmaceutical nasal dosage form of dihydroergotamine comprises dihydroergotamine and at least one stabilizer selected from the group of vitamin E derivatives, wherein said dosage form contains total impurities not more than about 5% or 4% or 3% or 2% or 1% when evaluated for at least about 6 months at about 25° C. with at least about 60% relative humidity.

In another aspect of the above embodiments, the pharmaceutical nasal dosage form of dihydroergotamine comprises dihydroergotamine and at least one stabilizer selected from the group of vitamin E derivatives, wherein said dosage form contains total impurities not more than about 5% or 4% or 3% or 2% or 1% when evaluated for at least about 6 months at about 40° C. with at least about 75% relative humidity.

In another aspect of the above embodiments, the pharmaceutical nasal dosage form of dihydroergotamine comprises dihydroergotamine and at least one stabilizer is benzalkonium chloride, wherein said dosage form contains total impurities not more than about 5% or 4% or 3% or 2% or 1% when evaluated for at least about 6 months at about 2° C. to 8° C.

In another aspect of the above embodiments, the pharmaceutical nasal dosage form of dihydroergotamine comprises dihydroergotamine and at least one stabilizer is benzalkonium chloride, wherein said dosage form contains total impurities not more than about 5% or 4% or 3% or 2% or 1% when evaluated for at least about 6 months at about 25° C. with at least about 60% relative humidity.

In another aspect of the above embodiments, the pharmaceutical nasal dosage form of dihydroergotamine comprises dihydroergotamine and at least one stabilizer is benzalkonium chloride, wherein said dosage form contains total impurities not more than about 5% or 4% or 3% or 2% or 1% when evaluated for at least about 6 months at about 40° C. with at least about 75% relative humidity.

In another aspect of the above embodiments, the pharmaceutical nasal dosage form of dihydroergotamine comprises dihydroergotamine and stabilizers selected from the group of sodium citrate, citric acid or combinations thereof in a weight ratio of from about 1.0:30.0 to about 30.0:1.0.

In another aspect of the above embodiments, the pharmaceutical nasal dosage form of dihydroergotamine comprises dihydroergotamine and sodium citrate in a weight ratio of from about 1.0:30.0 to about 30.0:1.0.

In another aspect of the above embodiments, the pharmaceutical nasal dosage form of dihydroergotamine comprises dihydroergotamine and sodium citrate in a weight ratio of from about 1.0:25.0 to about 25.0:1.0.

In another aspect of the above embodiments, the pharmaceutical nasal dosage form of dihydroergotamine comprises dihydroergotamine and stabilizers selected from the group of ammonium acetate, acetic acid or combinations thereof in a weight ratio of from about 1.0:40.0 to about 40.0:1.0.

In another aspect of the above embodiments, the pharmaceutical nasal dosage form of dihydroergotamine comprises dihydroergotamine and ammonium acetate in a weight ratio of from about 1.0:40.0 to about 40.0:1.0.

In another aspect of the above embodiments, the pharmaceutical nasal dosage form of dihydroergotamine comprises dihydroergotamine and ammonium acetate in a weight ratio of from about 1.0:30.0 to about 30.0:1.0.

In another aspect of the above embodiments, the pharmaceutical nasal dosage form of dihydroergotamine comprises dihydroergotamine and acid in a weight ratio of from about 1.0:30.0 to about 30.0:1.0.

In another aspect of the above embodiments, the pharmaceutical nasal dosage form of dihydroergotamine comprises dihydroergotamine and citric acid in a weight ratio of from about 1.0:30.0 to about 30.0:1.0.

In another aspect of the above embodiments, the pharmaceutical nasal dosage form of dihydroergotamine comprises dihydroergotamine and citric acid in a weight ratio of from about 1.0:25.0 to about 25.0:1.0.

In another aspect of the above embodiments, the pharmaceutical nasal dosage form of dihydroergotamine comprises dihydroergotamine and acetic acid in a weight ratio of from about 1.0:80.0 to about 80.0:1.0.

In another aspect of the above embodiments, the pharmaceutical nasal dosage form of dihydroergotamine comprises dihydroergotamine and acetic acid in a weight ratio of from about 1.0:50.0 to about 50.0:1.0.

In another aspect of the above embodiments, the pharmaceutical nasal dosage form of dihydroergotamine comprises dihydroergotamine and ascorbic acid in a weight ratio of from about 1.0:40.0 to about 40.0:1.0.

In another aspect of the above embodiments, the pharmaceutical nasal dosage form of dihydroergotamine comprises dihydroergotamine and ascorbic acid in a weight ratio of from about 1.0:30.0 to about 30.0:1.0.

In another aspect of the above embodiments, the pharmaceutical nasal dosage form of dihydroergotamine comprises dihydroergotamine and at least one stabilizer selected from one or more vitamin E derivatives in a weight ratio of from about 1.0:30.0 to about 30.0:1.0.

In another aspect of the above embodiments, the pharmaceutical nasal dosage form of dihydroergotamine comprises dihydroergotamine and benzalkonium chloride in a weight ratio of from about 1.0:80.0 to about 80.0:1.0.

In another aspect of the above embodiments, the pharmaceutical nasal dosage form of dihydroergotamine comprises dihydroergotamine and benzalkonium chloride in a weight ratio of from about 1.0:80.0 to about 80.0:1.0.

In another aspect of the above embodiments, the pharmaceutical nasal dosage form of dihydroergotamine comprises dihydroergotamine and vitamin E TPGS in a weight ratio of from about 1.0:30.0 to about 30.0:1.0.

In another aspect of the above embodiments, the pharmaceutical nasal dosage form of dihydroergotamine comprises dihydroergotamine and vitamin E TPGS in a weight ratio of from about 1.0:25.0 to about 25.0:1.0.

In another aspect of the above embodiments, the pharmaceutical nasal dosage form of dihydroergotamine comprises dihydroergotamine or a pharmaceutically acceptable salt thereof, one or more stabilizers and at least one pharmaceutically acceptable excipient, wherein said dosage form requires less than about 15 minutes for administration and minimizes the number of sprays to less than four sprays to administer effective dose of dihydroergotamine, wherein said effective dose is less than about 2 mg of dihydroergotamine.

In another aspect of the above embodiments, the pharmaceutical nasal dosage form of dihydroergotamine comprises dihydroergotamine or a pharmaceutically acceptable salt thereof, one or more stabilizers and at least one pharmaceutically acceptable excipient, wherein said dosage form requires less than about 15 minutes for administration and minimizes the number of sprays to less than three sprays to administer effective dose of dihydroergotamine, wherein said effective dose is less than about 2 mg of dihydroergotamine.

In another aspect of the above embodiments, the pharmaceutical nasal dosage form of dihydroergotamine comprises dihydroergotamine or a pharmaceutically acceptable salt thereof, one or more stabilizers and at least one pharmaceutically acceptable excipient, wherein said dosage form requires less than about 15 minutes for administration and minimizes the number of sprays to less than two sprays to administer effective dose of dihydroergotamine, wherein said effective dose is less than about 2 mg of dihydroergotamine.

In another aspect of the above embodiments, the pharmaceutical nasal dosage form of dihydroergotamine comprises dihydroergotamine or a pharmaceutically acceptable salt thereof, one or more stabilizers and at least one pharmaceutically acceptable excipient, wherein said dosage form requires less than about 15 minutes for administration and requires at least one spray to administer effective dose of dihydroergotamine, wherein said effective dose is less than about 2 mg of dihydroergotamine.

In another aspect of the above embodiments, the pharmaceutical nasal dosage form of dihydroergotamine comprises dihydroergotamine or a pharmaceutically acceptable salt thereof, one or more stabilizers and at least one pharmaceutically acceptable excipient, wherein said dosage form requires less than about 15 minutes for administration and minimizes the number of sprays to less than four sprays to administer effective dose of dihydroergotamine, wherein said effective dose is about 1.6 mg of dihydroergotamine.

In another aspect of the above embodiments, the pharmaceutical nasal dosage form of dihydroergotamine comprises dihydroergotamine or a pharmaceutically acceptable salt thereof, one or more stabilizers and at least one pharmaceutically acceptable excipient, wherein said dosage form requires less than about 15 minutes for administration and minimizes the number of sprays to less than three sprays to administer effective dose of dihydroergotamine, wherein said effective dose is about 1.6 mg of dihydroergotamine.

In another aspect of the above embodiments, the pharmaceutical nasal dosage form of dihydroergotamine comprises dihydroergotamine or a pharmaceutically acceptable salt thereof, one or more stabilizers and at least one pharmaceutically acceptable excipient, wherein said dosage form requires less than about 15 minutes for administration and minimizes the number of sprays to less than two sprays to administer effective dose of dihydroergotamine, wherein said effective dose is about 1.6 mg of dihydroergotamine.

In another aspect of the above embodiments, the pharmaceutical nasal dosage form of dihydroergotamine comprises dihydroergotamine or a pharmaceutically acceptable salt thereof, one or more stabilizers and at least one pharmaceutically acceptable excipient, wherein said dosage form requires less than about 15 minutes for administration and requires not more than two sprays to administer effective dose of dihydroergotamine, wherein said effective dose is about 1.6 mg of dihydroergotamine.

In another aspect of the above embodiments, the pharmaceutical nasal dosage form of dihydroergotamine comprises dihydroergotamine or a pharmaceutically acceptable salt thereof, one or more stabilizers and at least one pharmaceutically acceptable excipient, wherein said dosage form requires less than about 15 minutes for administration and requires at least one spray to administer effective dose of dihydroergotamine, wherein said effective dose is about 1.6 mg of dihydroergotamine.

In one aspect of the above embodiments, the pharmaceutical nasal dosage form of dihydroergotamine comprises dihydroergotamine or a pharmaceutically acceptable salt thereof, one or more stabilizers and at least one pharmaceutically acceptable excipient, wherein said dosage form requires less than about 15 minutes for administration and minimizes the number of sprays to less than four sprays to administer effective dose of dihydroergotamine, wherein said effective dose is about 1.3 mg of dihydroergotamine.

In another aspect of the above embodiments, the pharmaceutical nasal dosage form of dihydroergotamine comprises dihydroergotamine or a pharmaceutically acceptable salt thereof, one or more stabilizers and at least one pharmaceutically acceptable excipient, wherein said dosage form requires less than about 15 minutes for administration and minimizes the number of sprays to less than three sprays to administer effective dose of dihydroergotamine, wherein said effective dose is about 1.3 mg of dihydroergotamine.

In another aspect of the above embodiments, the pharmaceutical nasal dosage form of dihydroergotamine comprises dihydroergotamine or a pharmaceutically acceptable salt thereof, one or more stabilizers and at least one pharmaceutically acceptable excipient, wherein said dosage form requires less than about 15 minutes for administration and minimizes the number of sprays to less than two sprays to administer effective dose of dihydroergotamine, wherein said effective dose is about 1.3 mg of dihydroergotamine.

In another aspect of the above embodiments, the pharmaceutical nasal dosage form of dihydroergotamine comprises dihydroergotamine or a pharmaceutically acceptable salt thereof, one or more stabilizers and at least one pharmaceutically acceptable excipient, wherein said dosage form requires less than about 15 minutes for administration and requires not more than two sprays to administer effective dose of dihydroergotamine, wherein said effective dose is about 1.3 mg of dihydroergotamine.

In another aspect of the above embodiments, the pharmaceutical nasal dosage form of dihydroergotamine comprises dihydroergotamine or a pharmaceutically acceptable salt thereof, one or more stabilizers and at least one pharmaceutically acceptable excipient, wherein said dosage form requires less than about 15 minutes for administration and requires at least one spray to administer effective dose of dihydroergotamine, wherein said effective dose is about 1.3 mg of dihydroergotamine.

In one aspect of the above embodiments, the pharmaceutical nasal dosage form of dihydroergotamine comprises dihydroergotamine or a pharmaceutically acceptable salt thereof, one or more stabilizers and at least one pharmaceutically acceptable excipient, wherein said dosage form requires less than about 15 minutes for administration and minimizes the number of sprays to less than four sprays to administer effective dose of dihydroergotamine, wherein said effective dose is about 1.2 mg of dihydroergotamine.

In another aspect of the above embodiments, the pharmaceutical nasal dosage form of dihydroergotamine comprises dihydroergotamine or a pharmaceutically acceptable salt thereof, one or more stabilizers and at least one pharmaceutically acceptable excipient, wherein said dosage form requires less than about 15 minutes for administration and minimizes the number of sprays to less than three sprays to administer effective dose of dihydroergotamine, wherein said effective dose is about 1.2 mg of dihydroergotamine.

In another aspect of the above embodiments, the pharmaceutical nasal dosage form of dihydroergotamine comprises dihydroergotamine or a pharmaceutically acceptable salt thereof, one or more stabilizers and at least one pharmaceutically acceptable excipient, wherein said dosage form requires less than about 15 minutes for administration and minimizes the number of sprays to less than two sprays to administer effective dose of dihydroergotamine, wherein said effective dose is about 1.2 mg of dihydroergotamine.

In another aspect of the above embodiments, the pharmaceutical nasal dosage form of dihydroergotamine comprises dihydroergotamine or a pharmaceutically acceptable salt thereof, one or more stabilizers and at least one pharmaceutically acceptable excipient, wherein said dosage form requires less than about 15 minutes for administration and requires not more than two sprays to administer effective dose of dihydroergotamine, wherein said effective dose is about 1.2 mg of dihydroergotamine.

In another aspect of the above embodiments, the pharmaceutical nasal dosage form of dihydroergotamine comprises dihydroergotamine or a pharmaceutically acceptable salt thereof, one or more stabilizers and at least one pharmaceutically acceptable excipient, wherein said dosage form requires less than about 15 minutes for administration and requires at least one spray to administer effective dose of dihydroergotamine, wherein said effective dose is about 1.2 mg of dihydroergotamine.

In another aspect of the above embodiments, the pharmaceutical nasal dosage form of dihydroergotamine comprises dihydroergotamine or a pharmaceutically acceptable salt thereof, one or more stabilizers and at least one pharmaceutically acceptable excipient, wherein said dosage form requires less than about 15 minutes for administration and minimizes the number of sprays to less than four sprays to administer effective dose of dihydroergotamine, wherein said effective dose is about 1.1 mg of dihydroergotamine.

In another aspect of the above embodiments, the pharmaceutical nasal dosage form of dihydroergotamine comprises dihydroergotamine or a pharmaceutically acceptable salt thereof, one or more stabilizers and at least one pharmaceutically acceptable excipient, wherein said dosage form requires less than about 15 minutes for administration and minimizes the number of sprays to less than three sprays to administer effective dose of dihydroergotamine, wherein said effective dose is about 1.1 mg of dihydroergotamine.

In another aspect of the above embodiments, the pharmaceutical nasal dosage form of dihydroergotamine comprises dihydroergotamine or a pharmaceutically acceptable salt thereof, one or more stabilizers and at least one pharmaceutically acceptable excipient, wherein said dosage form requires less than about 15 minutes for administration and minimizes the number of sprays to less than two sprays to administer effective dose of dihydroergotamine, wherein said effective dose is about 1.1 mg of dihydroergotamine.

In another aspect of the above embodiments, the pharmaceutical nasal dosage form of dihydroergotamine comprises dihydroergotamine or a pharmaceutically acceptable salt thereof, one or more stabilizers and at least one pharmaceutically acceptable excipient, wherein said dosage form requires less than about 15 minutes for administration and requires not more than two sprays to administer effective dose of dihydroergotamine, wherein said effective dose is about 1.1 mg of dihydroergotamine.

In another aspect of the above embodiments, the pharmaceutical nasal dosage form of dihydroergotamine comprises dihydroergotamine or a pharmaceutically acceptable salt thereof, one or more stabilizers and at least one pharmaceutically acceptable excipient, wherein said dosage form requires less than about 15 minutes for administration and requires at least one spray to administer effective dose of dihydroergotamine, wherein said effective dose is about 1.1 mg of dihydroergotamine.

In another aspect of the above embodiments, the present application relates to a pharmaceutical nasal dosage form comprising aqueous solution of dihydroergotamine or a pharmaceutically acceptable salt thereof, in amount from about 0.5 mg/0.1 ml to about 2 mg/0.1 ml, one or more stabilizers in an amount from about 0.01% w/w to about 10% w/w and at least one pharmaceutically acceptable excipient for treating migraine with or without aura in human subjects, wherein said dosage form is administered using a pre-primed nasal device and said dosage form requires less than about 15 minutes for administration and minimizes the number of sprays to less than four sprays to administer effective dose of dihydroergotamine.

In another aspect of the above embodiments, the present application relates to a pharmaceutical nasal dosage form comprising aqueous solution of dihydroergotamine or a pharmaceutically acceptable salt thereof, in amount from about 0.5 mg/0.1 ml to about 2 mg/0.1 ml, one or more stabilizers in an amount from about 0.01% w/w to about 5% w/w and at least one pharmaceutically acceptable excipient for treating migraine with or without aura in in human subjects, wherein said dosage form is administered using a pre-primed nasal device and said dosage form requires less than about 15 minutes for administration and minimizes the number of sprays to less than four sprays to administer effective dose of dihydroergotamine.

In another aspect of the above embodiments, the present application relates to a pharmaceutical nasal dosage form comprising aqueous solution of dihydroergotamine or a pharmaceutically acceptable salt thereof, in amount from about 0.5 mg/0.1 ml to about 2 mg/0.1 ml, one or more stabilizers in an amount from about 0.01% w/w to about 2% w/w and at least one pharmaceutically acceptable excipient for treating migraine with or without aura in human subjects, wherein said dosage form is administered using a pre-primed nasal device and said dosage form requires less than about 15 minutes for administration and minimizes the number of sprays to less than four sprays to administer effective dose of dihydroergotamine.

In an aspect of the above embodiments, the administration of nasal sprays can be simultaneous or in a sequential order, to administer effective dose of dihydroergotamine.

In another aspect of the above embodiments, the pharmaceutical nasal dosage form of dihydroergotamine of the present application can be dispensed in a conventional nasal spray device meant for administering in the nasal cavity. Optionally the device may comprise one or more compressed inert gases, including but not limited to, CO₂, nitrogen or a hydrocarbon such as freon to provide the spray.

In another aspect of the above embodiments, the device may be constructed to receive a container to accommodate unit dosage or multiple dosages, e.g. an ampoule, capsule, vial or the like. For example the ampoule comprises sufficient volume, e.g. 0.05 ml to 5.0 mL, to provide single dose or several doses of the pharmaceutical nasal dosage form of dihydroergotamine.

In another aspect of the above embodiments, the pharmaceutical nasal dosage form of dihydroergotamine of the present application can be provided in the form of suspensions, emulsions, solutions, aerosols, powders, and the like.

In another aspect of the above embodiments, the pharmaceutical nasal dosage form of dihydroergotamine of the present application may be provided in a liquid form or in the form of dry powder. The liquid form can be solutions applied directly to the nasal cavity by conventional means, for example with a dropper, pipette or a spray or as solutions using pressurized metered-dose inhalers (pMDI), or as dry powders using dry powder inhaler devices (DPIs). Alternatively the formulation may also be administered by breath actuated inhalers (BDIs). The dry powder form can be a spray dried composition or a freeze dried composition having the drug in a micronized form and alternatively the drug can be in a microparticulate or a nanoparticulate form.

In another aspect of the above embodiments, the pharmaceutical nasal dosage form of dihydroergotamine of the present application optionally comprises one or more pharmaceutically acceptable excipients that are generally known in the art for nasal composition. Such excipients include, but are not limited to, solubilizers, preservatives, permeation enhancers, anti-oxidants, buffers, viscosity enhancing agents, osmotic agents, and like or combinations thereof.

Suitable solubilizers used in the present application include, but not limited to, xanthines or xanthine derivatives such as theophylline, caffeine, theobromine, aminophylline, paraxanthine, pentoxifylline and the like, propylene glycol, polyethylene glycols having a molecular weight between 400 and 1000, glycerin, C₂ to C₈ mono- and poly-alcohols (e.g., ethanol), C₇ to C₁₈ alcohols of linear or branched configuration, mixtures or combinations thereof.

Suitable preservatives used in the present application include, but not limited to, benzethonium chloride, methyl p-hydroxybenzoate, ethyl p-hydroxybenzoate, butyl p-hydroxybenzoate, propyl p-hydroxybenzoate, thimerosal, sodium dehydroacetate and myristyl-gamma-picolinium chloride, sodium benzoate, potassium benzoate, potassium sorbates and combinations thereof.

Suitable permeation enhancers used in the present application include, but not limited to, fatty acids of from 10 to 20 carbon atoms, such as oleic acid, lauric acid, myristic acid, stearic acid, linoleic acid, and palmitic acid; alkyl glycoside or saccharide alkyl ester selected from (1-O-n-Dodecyl-β-D-Maltopyranoside), tridecyl maltoside, sucrose monododecanoate, sucrose monotridecanoate and sucrose monotetradecanoate. mono-, di- and tri-glycerides of C₁₀₋₂₀ fatty acids, such as glycerol monolaurate, glycerol monooleate, glycerol dioleate, glycerol trioleate and glycerol monolinoleate; C₁₀₋₂₀ fatty acid mono-, di- and tri-esters of sorbitols, such as sorbitan monooleate, sorbitan trioleate, and sorbitan monolaurate; isopropyl myristate; sucrose monococoate; and the like; cyclodextrin, beta cyclodextrin; polyols such as polyethylene glycol, propylene glycol and combinations thereof.

Suitable viscosity enhancing agents used in the present application include, but not limited to polyvinyl alcohol, polyvinyl pyrrolidone, methyl cellulose, hydroxy propyl methylcellulose, hydroxyethyl cellulose, carboxymethyl cellulose, hydroxy propyl cellulose and combinations thereof.

Suitable osmotic agents used in the present application include, but not limited to mannitol, dextrose, sucrose, sodium chloride, or sorbitol and the like.

In another aspect of above embodiments, the present application relates to a pharmaceutical nasal dosage form of dihydroergotamine for treating migraine with or without aura in human subjects, wherein said dosage form comprises dihydroergotamine from about 0.5 mg to about 2 mg and upon intranasal administration to human subjects provides plasma concentration of at least about 700 pg/ml in about less than about 45 minutes compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 1.6 mg of dihydroergotamine and upon intranasal administration to human subjects provides plasma concentration of at least about 700 pg/ml in about less than about 45 minutes compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 1.3 mg of dihydroergotamine and upon intranasal administration to human subjects provides plasma concentration of at least about 700 pg/ml in about less than about 45 minutes compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 1.2 mg of dihydroergotamine and upon intranasal administration to human subjects provides plasma concentration of at least about 700 pg/ml in about less than about 45 minutes compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 1.1 mg of dihydroergotamine and upon intranasal administration to human subjects provides plasma concentration of at least about 700 pg/ml in about less than about 45 minutes compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg to about 2 mg, one or more stabilizers in an amount of from about 0.01% w/w to about 10% w/w and upon intranasal administration to human subjects provides plasma concentration of at least about 700 pg/ml in about less than about 45 minutes compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg to about 2 mg, one or more stabilizers in an amount of from about 0.01% w/w to about 5% w/w and upon intranasal administration to human subjects provides plasma concentration of at least about 700 pg/ml in about less than about 45 minutes compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg to about 2 mg, one or more stabilizers in an amount of from about 0.01% w/w to about 2% w/w and upon intranasal administration to human subjects provides plasma concentration of at least about 700 pg/ml in about less than about 45 minutes compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg/0.1 ml to about 2 mg/0.1 ml and upon intranasal administration to human subjects provides plasma concentration of at least about 700 pg/ml in about less than about 45 minutes compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 0.8 mg/0.1 ml of dihydroergotamine and upon intranasal administration to human subjects provides plasma concentration of at least about 700 pg/ml in about less than about 45 minutes compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 0.65 mg/0.1 ml of dihydroergotamine and upon intranasal administration to human subjects provides plasma concentration of at least about 700 pg/ml in about less than about 45 minutes compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 0.6 mg/0.1 ml of dihydroergotamine and upon intranasal administration to human subjects provides plasma concentration of at least about 700 pg/ml in about less than about 45 minutes compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 0.55 mg/0.1 ml of dihydroergotamine and upon intranasal administration to human subjects provides plasma concentration of at least about 700 pg/ml in about less than about 45 minutes compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg/0.1 ml to about 2 mg/0.1 ml, one or more stabilizers in an amount of from about 0.01% w/w to about 10% w/w and upon intranasal administration to human subjects provides plasma concentration of at least about 700 pg/ml in about less than about 45 minutes compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg/0.1 ml to about 2 mg/0.1 ml, one or more stabilizers in an amount of from about 0.01% w/w to about 5% w/w and upon intranasal administration to human subjects provides plasma concentration of at least about 700 pg/ml in about less than about 45 minutes compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg/0.1 ml to about 2 mg/0.1 ml, one or more stabilizers in an amount of from about 0.01% w/w to about 2% w/w and upon intranasal administration to human subjects provides plasma concentration of at least about 700 pg/ml in about less than about 45 minutes compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg to about 2 mg and upon intranasal administration to human subjects provides at least about 10% reduction in time required to achieve plasma concentration of at least about 700 pg/ml compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 1.6 mg of dihydroergotamine and upon intranasal administration to human subjects provides at least about 10% reduction in time required to achieve plasma concentration of at least about 700 pg/ml compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 1.3 mg of dihydroergotamine and upon intranasal administration to human subjects provides at least about 10% reduction in time required to achieve plasma concentration of at least about 700 pg/ml compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 1.2 mg of dihydroergotamine and upon intranasal administration to human subjects provides at least about 10% reduction in time required to achieve plasma concentration of at least about 700 pg/ml compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 1.1 mg of dihydroergotamine and upon intranasal administration to human subjects provides at least about 10% reduction in time required to achieve plasma concentration of at least about 700 pg/ml compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg to about 2 mg, one or more stabilizers in an amount of from about 0.01% w/w to about 10% w/w and upon intranasal administration to human subjects provides at least about 10% reduction in time required to achieve plasma concentration of at least about 700 pg/ml compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg to about 2 mg, one or more stabilizers in an amount of from about 0.01% w/w to about 5% w/w and upon intranasal administration to human subjects provides at least about 10% reduction in time required to achieve plasma concentration of at least about 700 pg/ml compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg to about 2 mg, one or more stabilizers in an amount of from about 0.01% w/w to about 2% w/w and upon intranasal administration to human subjects provides at least about 10% reduction in time required to achieve plasma concentration of at least about 700 pg/ml compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg/0.1 ml to about 2 mg/0.1 ml and upon intranasal administration to human subjects provides at least about 10% reduction in time required to achieve plasma concentration of at least about 700 pg/ml compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 0.8 mg/0.1 ml of dihydroergotamine and upon intranasal administration to human subjects provides at least about 10% reduction in time required to achieve plasma concentration of at least about 700 pg/ml compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 0.65 mg/0.1 ml of dihydroergotamine and upon intranasal administration to human subjects provides at least about 10% reduction in time required to achieve plasma concentration of at least about 700 pg/ml compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 0.6 mg/0.lml of dihydroergotamine and upon intranasal administration to human subjects provides at least about 10% reduction in time required to achieve plasma concentration of at least about 700 pg/ml compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 0.55 mg/0.1 ml of dihydroergotamine and upon intranasal administration to human subjects provides at least about 10% reduction in time required to achieve plasma concentration of at least about 700 pg/ml compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg/0.1 ml to about 2 mg/0.1 ml, one or more stabilizers in an amount of from about 0.01% w/w to about 10% w/w and upon intranasal administration to human subjects provides at least about 10% reduction in time required to achieve plasma concentration of at least about 700 pg/ml compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg/0.1 ml to about 2 mg/0.1 ml, one or more stabilizers in an amount of from about 0.01% w/w to about 5% w/w and upon intranasal administration to human subjects provides at least about 10% reduction in time required to achieve plasma concentration of at least about 700 pg/ml compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg/0.1 ml to about 2 mg/0.1 ml, one or more stabilizers in an amount of from about 0.01% w/w to about 2% w/w and upon intranasal administration to human subjects provides at least about 10% reduction in time required to achieve plasma concentration of at least about 700 pg/ml compared to a commercially-available 2 mg dihydroergotamine nasal dosage form.

In another aspect of above embodiments, the reduction in time required to achieve plasma concentration of at least about 700 pg/ml compared to a commercially-available 2 mg dihydroergotamine nasal dosage form is at least about 10% or 15% or 20% or 25% or 30% or 35% or 40% or 45% or 50% or 55% or 60% or 65% or 70% or 75% or 80% or 85% or 90% or 95%.

In another aspect of above embodiments, the present application relates to a pharmaceutical nasal dosage form of dihydroergotamine for treating migraine with or without aura in human subjects, wherein said dosage form comprises dihydroergotamine from about 0.5 mg to about 2 mg and upon intranasal administration to human subjects provides a higher Cₘₐₓ compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg/0.1 ml to about 2 mg/0.1 ml and upon intranasal administration to human subjects provides at least about a 10 percent higher Cₘₐₓ compared to a commercially-available 2 mg dihydroergotamine nasal dosage form.

In another aspect of above embodiments, the present application relates to pharmaceutical nasal dosage form comprising aqueous solution of dihydroergotamine or a pharmaceutically acceptable salt thereof, for treating migraine with or without aura in human subjects, wherein said dosage form comprises dihydroergotamine from about 0.5 mg/0.1 ml to about 2 mg/0.1 ml and upon intranasal administration to human subjects provides at least about a 10 percent higher Cₘₐₓ compared to a commercially-available 2 mg dihydroergotamine nasal dosage form.

In another aspect of above embodiments, the present application relates to pharmaceutical nasal dosage form comprising aqueous solution of dihydroergotamine or a pharmaceutically acceptable salt thereof, for treating migraine with or without aura in human subjects, wherein said dosage form comprises about 0.8 mg/0.1 ml of dihydroergotamine and upon intranasal administration to human subjects provides at least about a 10 percent higher Cₘₐₓ compared to a commercially-available 2 mg dihydroergotamine nasal dosage form.

In another aspect of above embodiments, the present application relates to pharmaceutical nasal dosage form comprising aqueous solution of dihydroergotamine or a pharmaceutically acceptable salt thereof, for treating migraine with or without aura in human subjects, wherein said dosage form comprises about 0.65 mg/0.1 ml of dihydroergotamine and upon intranasal administration to human subjects provides at least about a 10 percent higher Cₘₐₓ compared to a commercially-available 2 mg dihydroergotamine nasal dosage form.

In another aspect of above embodiments, the present application relates to pharmaceutical nasal dosage form comprising aqueous solution of dihydroergotamine or a pharmaceutically acceptable salt thereof, for treating migraine with or without aura in human subjects, wherein said dosage form comprises about 0.6 mg/0.1 ml of dihydroergotamine and upon intranasal administration to human subjects provides at least about a 10 percent higher Cₘₐₓ compared to a commercially-available 2 mg dihydroergotamine nasal dosage form.

In another aspect of above embodiments, the present application relates to pharmaceutical nasal dosage form comprising aqueous solution of dihydroergotamine or a pharmaceutically acceptable salt thereof, for treating migraine with or without aura in human subjects, wherein said dosage form comprises about 0.55 mg/0.1 ml of dihydroergotamine and upon intranasal administration to human subjects provides at least about a 10 percent higher Cₘₐₓ compared to a commercially-available 2 mg dihydroergotamine nasal dosage form.

In another embodiment, the present application relates to pharmaceutical nasal dosage form comprising aqueous solution of dihydroergotamine or a pharmaceutically acceptable salt thereof, for treating migraine with or without aura in human subjects, wherein said dosage form comprises dihydroergotamine from about 0.5 mg/0.1 ml to about 2 mg/0.1 ml, one or more stabilizers in an amount of from about 0.01% w/w to about 10% w/w and at least one pharmaceutically acceptable excipient and upon intranasal administration to human subjects provides at least about a 10 percent higher Cₘₐₓ compared to a commercially-available 2 mg dihydroergotamine nasal dosage form.

In another embodiment, the present application relates to pharmaceutical nasal dosage form comprising aqueous solution of dihydroergotamine or a pharmaceutically acceptable salt thereof, for treating migraine with or without aura in human subjects, wherein said dosage form comprises dihydroergotamine from about 0.5 mg/0.1 ml to about 2 mg/0.1 ml, one or more stabilizers in an amount of from about 0.01% w/w to about 5% w/w and at least one pharmaceutically acceptable excipient and upon intranasal administration to human subjects provides at least about a 10 percent higher Cₘₐₓ compared to a commercially-available 2 mg dihydroergotamine nasal dosage form.

In another embodiment, the present application relates to pharmaceutical nasal dosage form comprising aqueous solution of dihydroergotamine or a pharmaceutically acceptable salt thereof, for treating migraine with or without aura in human subjects, wherein said dosage form comprises dihydroergotamine from about 0.5 mg/0.1 ml to about 2 mg/0.1 ml, one or more stabilizers in an amount of from about 0.01% w/w to about 2% w/w and at least one pharmaceutically acceptable excipient and upon intranasal administration to human subjects provides at least about a 10 percent higher Cₘₐₓ compared to a commercially-available 2 mg dihydroergotamine nasal dosage form.

In another embodiment the present application relates to a pharmaceutical nasal dosage form of dihydroergotamine for treating migraine with or without aura in human subjects, wherein said dosage form comprises dihydroergotamine from about 0.5 mg/0.1 ml to about 2 mg/0.1 ml and upon intranasal administration to human subjects provides at least about a 20 percent higher Cₘₐₓ compared to a commercially-available 2 mg dihydroergotamine nasal dosage form.

In another embodiment the present application relates to a pharmaceutical nasal dosage form of dihydroergotamine for treating migraine with or without aura in human subjects, wherein said dosage form comprises dihydroergotamine from about 0.5 mg/0.1 ml to about 2 mg/0.1 ml and upon intranasal administration to human subjects provides at least about 30 percent higher Cₘₐₓ compared to a commercially-available 2 mg dihydroergotamine nasal dosage form.

In certain aspects of above embodiments, the pharmaceutical nasal dosage form of dihydroergotamine of present application, upon intranasal administration to human subjects provide Cₘₐₓ of at least about 700 pg*hr/mL.

In another aspect of above embodiments, the present application relates to a pharmaceutical nasal dosage form of dihydroergotamine for treating migraine with or without aura in human subjects, wherein said dosage form comprises dihydroergotamine from about 0.5 mg to about 2 mg and upon intranasal administration to human subjects provides at least about 10% reduction in coefficient of variance (CV %) of Cₘₐₓ compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 1.6 mg of dihydroergotamine and upon intranasal administration to human subjects provides at least about 10% reduction in coefficient of variance (CV %) of Cₘₐₓ compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 1.3 mg of dihydroergotamine and upon intranasal administration to human subjects provides at least about 10% reduction in coefficient of variance (CV %) of Cₘₐₓ compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 1.2 mg of dihydroergotamine and upon intranasal administration to human subjects provides at least about 10% reduction in coefficient of variance (CV %) of Cₘₐₓ compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 1.1 mg of dihydroergotamine and upon intranasal administration to human subjects provides at least about 10% reduction in coefficient of variance (CV %) of Cₘₐₓ compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg/0.1 ml to about 2 mg/0.1 ml and upon intranasal administration to human subjects provides at least about 10% reduction in coefficient of variance (CV %) of Cₘₐₓ compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 0.8 mg/0.1 ml of dihydroergotamine and upon intranasal administration to human subjects provides at least about 10% reduction in coefficient of variance (CV %) of Cₘₐₓ compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 0.65 mg/ml of dihydroergotamine and upon intranasal administration to human subjects provides at least about 10% reduction in coefficient of variance (CV %) of Cₘₐₓ compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 0.6 mg/0.1 ml of dihydroergotamine and upon intranasal administration to human subjects provides at least about 10% reduction in coefficient of variance (CV %) of Cₘₐₓ compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 0.55 mg/0.1 ml of dihydroergotamine and upon intranasal administration to human subjects provides at least about 10% reduction in coefficient of variance (CV %) of Cₘₐₓ compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg to about 2 mg and upon intranasal administration to human subjects provide higher AUC_{(0-2 hr)} compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg/0.1 ml to about 2 mg/0.1 ml and upon intranasal administration to human subjects provides at least about a 10 percent higher AUC_{(0-2 hr)} compared to a commercially-available 2 mg dihydroergotamine nasal dosage form.

In another aspect of above embodiments, the present application relates to pharmaceutical nasal dosage form comprising aqueous solution of dihydroergotamine or a pharmaceutically acceptable salt thereof, for treating migraine with or without aura in human subjects, wherein said dosage form comprises dihydroergotamine from about 0.5 mg/0.1 ml to about 2 mg/0.1 ml and upon intranasal administration to human subjects provides at least about a 10 percent higher AUC_{(0-2 hr)} compared to a commercially-available 2 mg dihydroergotamine nasal dosage form.

In another aspect of above embodiments, the present application relates to pharmaceutical nasal dosage form comprising aqueous solution of dihydroergotamine or a pharmaceutically acceptable salt thereof, for treating migraine with or without aura in human subjects, wherein said dosage form comprises about 0.8 mg/0.1 ml of dihydroergotamine and upon intranasal administration to human subjects provides at least about a 10 percent higher AUC_{(0-2 hr )} compared to a commercially-available 2 mg dihydroergotamine nasal dosage form.

In another aspect of above embodiments, the present application relates to pharmaceutical nasal dosage form comprising aqueous solution of dihydroergotamine or a pharmaceutically acceptable salt thereof, for treating migraine with or without aura in human subjects, wherein said dosage form comprises about 0.65 mg/0.1 ml of dihydroergotamine and upon intranasal administration to human subjects provides at least about a 10 percent higher AUC_{(0-2 hr)} compared to a commercially-available 2 mg dihydroergotamine nasal dosage form.

In another aspect of above embodiments, the present application relates to pharmaceutical nasal dosage form comprising aqueous solution of dihydroergotamine or a pharmaceutically acceptable salt thereof, for treating migraine with or without aura in human subjects, wherein said dosage form comprises about 0.6 mg/0.1 ml of dihydroergotamine and upon intranasal administration to human subjects provides at least about a 10 percent higher AUC_{(0-2 hr)} compared to a commercially-available 2 mg dihydroergotamine nasal dosage form.

In another aspect of above embodiments, the present application relates to pharmaceutical nasal dosage form comprising aqueous solution of dihydroergotamine or a pharmaceutically acceptable salt thereof, for treating migraine with or without aura in human subjects, wherein said dosage form comprises about 0.55 mg/0.1 ml of dihydroergotamine and upon intranasal administration to human subjects provides at least about a 10 percent higher AUC_{(0-2 hr )} compared to a commercially-available 2 mg dihydroergotamine nasal dosage form.

In another embodiment, the present application relates to pharmaceutical nasal dosage form comprising aqueous solution of dihydroergotamine or a pharmaceutically acceptable salt thereof, for treating migraine with or without aura in human subjects, wherein said dosage form comprises dihydroergotamine from about 0.5 mg/0.1 ml to about 2 mg/0.1 ml, one or more stabilizers in an amount of from about 0.01% w/w to about 10% w/w and at least one pharmaceutically acceptable excipient and upon intranasal administration to human subjects provides at least about a 10 percent higherAUC_{(0-2 hr)} compared to a commercially-available 2 mg dihydroergotamine nasal dosage form.

In another embodiment, the present application relates to pharmaceutical nasal dosage form comprising aqueous solution of dihydroergotamine or a pharmaceutically acceptable salt thereof, for treating migraine with or without aura in human subjects, wherein said dosage form comprises dihydroergotamine from about 0.5 mg/0.1 ml to about 2 mg/0.1 ml, one or more stabilizers in an amount of from about 0.01% w/w to about 5% w/w and at least one pharmaceutically acceptable excipient and upon intranasal administration to human subjects provides at least about a 10 percent higherAUC_{(0-2 hr)} compared to a commercially-available 2 mg dihydroergotamine nasal dosage form.

In another embodiment, the present application relates to pharmaceutical nasal dosage form comprising aqueous solution of dihydroergotamine or a pharmaceutically acceptable salt thereof, for treating migraine with or without aura in human subjects, wherein said dosage form comprises dihydroergotamine from about 0.5 mg/0.1 ml to about 2 mg/0.1 ml, one or more stabilizers in an amount of from about 0.01% w/w to about 2% w/w and at least one pharmaceutically acceptable excipient and upon intranasal administration to human subjects provides at least about a 10 percent higherAUC_{(0-2 hr)} compared to a commercially-available 2 mg dihydroergotamine nasal dosage form.

In another embodiment the present application relates to a pharmaceutical nasal dosage form of dihydroergotamine for treating migraine with or without aura in human subjects, wherein said dosage form comprises dihydroergotamine from about 0.5 mg/0.1 ml to about 2 mg/0.1 ml and upon intranasal administration to human subjects provides at least about a 20 percent higher AUC_{(0-2 hr)} compared to a commercially-available 2 mg dihydroergotamine nasal dosage form.

In another embodiment the present application relates to a pharmaceutical nasal dosage form of dihydroergotamine for treating migraine with or without aura in human subjects, wherein said dosage form comprises dihydroergotamine from about 0.5 mg/0.1 ml to about 2 mg/0.1 ml and upon intranasal administration to human subjects provides at least about 30 percent higher AUC_{(0-2 hr)} compared to a commercially-available 2 mg dihydroergotamine nasal dosage form.

In another embodiment the present application relates to a pharmaceutical nasal dosage form of dihydroergotamine for treating migraine with or without aura in human subjects, wherein said dosage form comprises dihydroergotamine from about 0.5 mg/0.1 ml to about 2 mg/0.1 ml and upon intranasal administration to human subjects provides at least about 40 percent higher AUC_{(0-2 hr)} compared to a commercially-available 2 mg dihydroergotamine nasal dosage form.

In certain aspects of above embodiments, the pharmaceutical nasal dosage form of dihydroergotamine of present application, upon intranasal administration to human subjects provideAUC_{(0-2 hr)} of at least about 1200 pg*hr/mL.

In another aspect of above embodiments, the present application relates to a pharmaceutical nasal dosage form of dihydroergotamine for treating migraine with or without aura in human subjects, wherein said dosage form comprises dihydroergotamine from about 0.5 mg to about 2 mg and upon intranasal administration to human subjects provides at least about 10% reduction in coefficient of variance (CV %) of AUC_{(0-2 hr)} compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 1.6 mg of dihydroergotamine and upon intranasal administration to human subjects provides at least about 10% reduction in coefficient of variance (CV %) of AUC_{(0-2 hr)} compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 1.3 mg of dihydroergotamine and upon intranasal administration to human subjects provides at least about 10% reduction in coefficient of variance (CV %) of AUC_{(0-2 hr)} compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 1.2 mg of dihydroergotamine and upon intranasal administration to human subjects provides at least about 10% reduction in coefficient of variance (CV %) of AUC_{(0-2 hr)} compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 1.1 mg of dihydroergotamine and upon intranasal administration to human subjects provides at least about 10% reduction in coefficient of variance (CV %) of AUC_{(0-2 hr)} compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg/0.1 ml to about 2 mg/0.1 ml and upon intranasal administration to human subjects provides at least about 10% reduction in coefficient of variance (CV %) of AUC_{(0-2 hr)} compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 0.8 mg/0.1 ml of dihydroergotamine and upon intranasal administration to human subjects provides at least about 10% reduction in coefficient of variance (CV %) of AUC_{(0-2 hr )} compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 0.65 mg/0.1 ml of dihydroergotamine and upon intranasal administration to human subjects provides at least about 10% reduction in coefficient of variance (CV %) of AUC_{(0-2 hr)} compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 0.6 mg/0.1 ml of dihydroergotamine and upon intranasal administration to human subjects provides at least about 10% reduction in coefficient of variance (CV %) of AUC_{(0-2 hr)} compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 0.55 mg/0.1 ml of dihydroergotamine and upon intranasal administration to human subjects provides at least about 10% reduction in coefficient of variance (CV %) of AUC_{(0-2 hr)} compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg to about 2 mg and upon intranasal administration to human subjects provide higher AUC_{(0-2 hr)} compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg/0.1 ml to about 2 mg/0.1 ml and upon intranasal administration to human subjects provides at least about a 10 percent higher AUC_{(0-2 hr)} compared to a commercially-available 2 mg dihydroergotamine nasal dosage form.

In another aspect of above embodiments, the present application relates to pharmaceutical nasal dosage form comprising aqueous solution of dihydroergotamine or a pharmaceutically acceptable salt thereof, for treating migraine with or without aura in human subjects, wherein said dosage form comprises dihydroergotamine from about 0.5 mg/0.1 ml to about 2 mg/0.1 ml and upon intranasal administration to human subjects provides at least about a 10 percent higher AUC_{(0-2 hr)} compared to a commercially-available 2 mg dihydroergotamine nasal dosage form.

In another aspect of above embodiments, the present application relates to pharmaceutical nasal dosage form comprising aqueous solution of dihydroergotamine or a pharmaceutically acceptable salt thereof, for treating migraine with or without aura in human subjects, wherein said dosage form comprises about 0.8 mg/0.1 ml of dihydroergotamine and upon intranasal administration to human subjects provides at least about a 10 percent higher AUC₍₀₋ₜ₎ compared to a commercially-available 2 mg dihydroergotamine nasal dosage form.

In another aspect of above embodiments, the present application relates to pharmaceutical nasal dosage form comprising aqueous solution of dihydroergotamine or a pharmaceutically acceptable salt thereof, for treating migraine with or without aura in human subjects, wherein said dosage form comprises about 0.65 mg/0.1 ml of dihydroergotamine and upon intranasal administration to human subjects provides at least about a 10 percent higher AUC₍₀₋ₜ₎ compared to a commercially-available 2 mg dihydroergotamine nasal dosage form.

In another aspect of above embodiments, the present application relates to pharmaceutical nasal dosage form comprising aqueous solution of dihydroergotamine or a pharmaceutically acceptable salt thereof, for treating migraine with or without aura in human subjects, wherein said dosage form comprises about 0.6 mg/0.1 ml of dihydroergotamine and upon intranasal administration to human subjects provides at least about a 10 percent higher AUC₍₀₋ₜ₎ compared to a commercially-available 2 mg dihydroergotamine nasal dosage form.

In another aspect of above embodiments, the present application relates to pharmaceutical nasal dosage form comprising aqueous solution of dihydroergotamine or a pharmaceutically acceptable salt thereof, for treating migraine with or without aura in human subjects, wherein said dosage form comprises about 0.55 mg/0.1 ml of dihydroergotamine and upon intranasal administration to human subjects provides at least about a 10 percent higher AUC₍₀₋ₜ₎ compared to a commercially-available 2 mg dihydroergotamine nasal dosage form.

In another embodiment, the present application relates to pharmaceutical nasal dosage form comprising aqueous solution of dihydroergotamine or a pharmaceutically acceptable salt thereof, for treating migraine with or without aura in human subjects, wherein said dosage form comprises dihydroergotamine from about 0.5 mg/0.1 ml to about 2 mg/0.1 ml, one or more stabilizers in an amount of from about 0.01% w/w to about 10% w/w and at least one pharmaceutically acceptable excipient and upon intranasal administration to human subjects provides at least about a 10 percent higher AUC₍₀₋ₜ₎ compared to a commercially-available 2 mg dihydroergotamine nasal dosage form.

In another embodiment, the present application relates to pharmaceutical nasal dosage form comprising aqueous solution of dihydroergotamine or a pharmaceutically acceptable salt thereof, for treating migraine with or without aura in human subjects, wherein said dosage form comprises dihydroergotamine from about 0.5 mg/0.1 ml to about 2 mg/0.1 ml, one or more stabilizers in an amount of from about 0.01% w/w to about 5% w/w and at least one pharmaceutically acceptable excipient and upon intranasal administration to human subjects provides at least about a 10 percent higher AUC₍₀₋ₜ₎ compared to a commercially-available 2 mg dihydroergotamine nasal dosage form.

In another embodiment, the present application relates to pharmaceutical nasal dosage form comprising aqueous solution of dihydroergotamine or a pharmaceutically acceptable salt thereof, for treating migraine with or without aura in human subjects, wherein said dosage form comprises dihydroergotamine from about 0.5 mg/0.1 ml to about 2 mg/0.1 ml, one or more stabilizers in an amount of from about 0.01% w/w to about 2% w/w and at least one pharmaceutically acceptable excipient and upon intranasal administration to human subjects provides at least about a 10 percent higher AUC₍₀₋ₜ₎ compared to a commercially-available 2 mg dihydroergotamine nasal dosage form.

In another embodiment the present application relates to a pharmaceutical nasal dosage form of dihydroergotamine for treating migraine with or without aura in human subjects, wherein said dosage form comprises dihydroergotamine from about 0.5 mg/0.1 ml to about 2 mg/0.1 ml and upon intranasal administration to human subjects provides at least about a 20 percent higher AUC₍₀₋ₜ₎ compared to a commercially-available 2 mg dihydroergotamine nasal dosage form.

In another embodiment the present application relates to a pharmaceutical nasal dosage form of dihydroergotamine for treating migraine with or without aura in human subjects, wherein said dosage form comprises dihydroergotamine from about 0.5 mg/0.1 ml to about 2 mg/0.1 ml and upon intranasal administration to human subjects provides at least about 30 percent higher AUC₍₀₋ₜ₎ compared to a commercially-available 2 mg dihydroergotamine nasal dosage form.

In another embodiment the present application relates to a pharmaceutical nasal dosage form of dihydroergotamine for treating migraine with or without aura in human subjects, wherein said dosage form comprises dihydroergotamine from about 0.5 mg/0.1 ml to about 2 mg/0.1 ml and upon intranasal administration to human subjects provides at least about 40 percent higher AUC₍₀₋ₜ₎ compared to a commercially-available 2 mg dihydroergotamine nasal dosage form.

In certain aspects of above embodiments, the pharmaceutical nasal dosage form of dihydroergotamine of present application, upon intranasal administration to human subjects provide AUC₍₀₋ₜ₎ of at least about 4500 pg*hr/mL.

In another aspect of above embodiments, the present application relates to a pharmaceutical nasal dosage form of dihydroergotamine for treating migraine with or without aura in human subjects, wherein said dosage form comprises dihydroergotamine from about 0.5 mg to about 2 mg and upon intranasal administration to human subjects provides at least about a 10% reduction in coefficient of variance (CV %) of AUC₍₀₋ₜ₎ compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 1.6 mg of dihydroergotamine and upon intranasal administration to human subjects provides at least about a 10% reduction in coefficient of variance (CV %) of AUC₍₀₋ₜ₎ compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 1.3 mg of dihydroergotamine and upon intranasal administration to human subjects provides at least about 10% reduction in coefficient of variance (CV %) of AUC₀₋ₜ₎ compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 1.2 mg of dihydroergotamine and upon intranasal administration to human subjects provides at least about 10% reduction in coefficient of variance (CV %) of AUC₍₀₋ₜ₎ compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 1.1 mg of dihydroergotamine and upon intranasal administration to human subjects provides at least about a 10% reduction in coefficient of variance (CV %) of AUC₍₀₋ₜ₎ compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg/0.1 ml to about 2 mg/0.1 ml and upon intranasal administration to human subjects provides at least about 10% reduction in coefficient of variance (CV %) of AUC₍₀₋ₜ₎ compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 0.8 mg/0.1 ml of dihydroergotamine and upon intranasal administration to human subjects provides at least about 10% reduction in coefficient of variance (CV %) of AUC₍₀₋ₜ₎ compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 0.65 mg/0.1 ml of dihydroergotamine and upon intranasal administration to human subjects provides at least about 10% reduction in coefficient of variance (CV %) of AUC₍₀₋ₜ₎ compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 0.6 mg/0.1 ml of dihydroergotamine and upon intranasal administration to human subjects provides at least about 10% reduction in coefficient of variance (CV %) of AUC₍₀₋ₜ₎ compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 0.55 mg/0.1 ml of dihydroergotamine and upon intranasal administration to human subjects provides at least about 10% reduction in coefficient of variance (CV %) of AUC₍₀₋ₜ₎ compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg to about 2 mg and upon intranasal administration to human subjects provide higher AUC_{(0-∞)} compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg/0.1 ml to about 2 mg/0.1 ml and upon intranasal administration to human subjects provides at least about a 10 percent higher AUC_{(0-∞)} compared to a commercially-available 2 mg dihydroergotamine nasal dosage form.

In another aspect of above embodiments, the present application relates to pharmaceutical nasal dosage form comprising aqueous solution of dihydroergotamine or a pharmaceutically acceptable salt thereof, for treating migraine with or without aura in human subjects, wherein said dosage form comprises dihydroergotamine from about 0.5 mg/0.1 ml to about 2 mg/0.1 ml and upon intranasal administration to human subjects provides at least about a 10 percent higher AUC_{(0-∞)} compared to a commercially-available 2 mg dihydroergotamine nasal dosage form.

In another aspect of above embodiments, the present application relates to pharmaceutical nasal dosage form comprising aqueous solution of dihydroergotamine or a pharmaceutically acceptable salt thereof, for treating migraine with or without aura in human subjects, wherein said dosage form comprises about 0.8 mg/0.1 ml of dihydroergotamine and upon intranasal administration to human subjects provides at least about a 10 percent higher AUC_{(0-∞)} compared to a commercially-available 2 mg dihydroergotamine nasal dosage form.

In another aspect of above embodiments, the present application relates to pharmaceutical nasal dosage form comprising aqueous solution of dihydroergotamine or a pharmaceutically acceptable salt thereof, for treating migraine with or without aura in human subjects, wherein said dosage form comprises about 0.65 mg/0.1 ml of dihydroergotamine and upon intranasal administration to human subjects provides at least about a 10 percent higher AUC_{(0-∞)} compared to a commercially-available 2 mg dihydroergotamine nasal dosage form.

In another aspect of above embodiments, the present application relates to pharmaceutical nasal dosage form comprising aqueous solution of dihydroergotamine or a pharmaceutically acceptable salt thereof, for treating migraine with or without aura in human subjects, wherein said dosage form comprises about 0.6 mg/0.1 ml of dihydroergotamine and upon intranasal administration to human subjects provides at least about a 10 percent higher AUC_{(0-∞)} compared to a commercially-available 2 mg dihydroergotamine nasal dosage form.

In another aspect of above embodiments, the present application relates to pharmaceutical nasal dosage form comprising aqueous solution of dihydroergotamine or a pharmaceutically acceptable salt thereof, for treating migraine with or without aura in human subjects, wherein said dosage form comprises about 0.55 mg/0.1 ml of dihydroergotamine and upon intranasal administration to human subjects provides at least about a 10 percent higher AUC_{(0-∞)} compared to a commercially-available 2 mg dihydroergotamine nasal dosage form.

In another aspect of above embodiments, the present application relates to pharmaceutical nasal dosage form comprising aqueous solution of dihydroergotamine or a pharmaceutically acceptable salt thereof, for treating migraine with or without aura in human subjects, wherein said dosage form comprises dihydroergotamine from about 0.5 mg/0.1 ml to about 2 mg/0.1 ml, one or more stabilizers in an amount of from about 0.01% w/w to about 10% w/w and at least one pharmaceutically acceptable excipient and upon intranasal administration to human subjects provides at least about a 10 percent higher AUC_{(0-∞)} compared to a commercially-available 2 mg dihydroergotamine nasal dosage form.

In another aspect of above embodiments, the present application relates to pharmaceutical nasal dosage form comprising aqueous solution of dihydroergotamine or a pharmaceutically acceptable salt thereof, for treating migraine with or without aura in human subjects, wherein said dosage form comprises dihydroergotamine from about 0.5 mg/0.1 ml to about 2 mg/0.1 ml, one or more stabilizers in an amount of from about 0.01% w/w to about 5% w/w and at least one pharmaceutically acceptable excipient and upon intranasal administration to human subjects provides at least about a 10 percent higher AUC_{(0-∞)} compared to a commercially-available 2 mg dihydroergotamine nasal dosage form.

In another aspect of above embodiments, the present application relates to pharmaceutical nasal dosage form comprising aqueous solution of dihydroergotamine or a pharmaceutically acceptable salt thereof, for treating migraine with or without aura in human subjects, wherein said dosage form comprises dihydroergotamine from about 0.5 mg/0.1 ml to about 2 mg/0.1 ml, one or more stabilizers in an amount of from about 0.01% w/w to about 2% w/w and at least one pharmaceutically acceptable excipient and upon intranasal administration to human subjects provides at least about a 10 percent higher AUC_{(0-∞)} compared to a commercially-available 2 mg dihydroergotamine nasal dosage form.

In certain aspects of above embodiments, the pharmaceutical nasal dosage form of dihydroergotamine of present application, upon intranasal administration to human subjects provide AUC_{(0-∞)} of at least about 5000 pg*hr/mL.

In another aspect of above embodiments, the present application relates to a pharmaceutical nasal dosage form of dihydroergotamine for treating migraine with or without aura in human subjects, wherein said dosage form comprises dihydroergotamine from about 0.5 mg to about 2 mg and upon intranasal administration to human subjects provides at least about 10% reduction in coefficient of variance (CV %) of AUC_{(0-∞)} compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 1.6 mg of dihydroergotamine and upon intranasal administration to human subjects provides at least about 10% reduction in coefficient of variance (CV %) of AUC_{(0-∞)} compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 1.3 mg of dihydroergotamine and upon intranasal administration to human subjects provides at least about 10% reduction in coefficient of variance (CV %) of AUC_{(0-∞)} compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 1.2 mg of dihydroergotamine and upon intranasal administration to human subjects provides at least about 10% reduction in coefficient of variance (CV %) of AUC_{(0-∞)} compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 1.1 mg of dihydroergotamine and upon intranasal administration to human subjects provides at least about 10% reduction in coefficient of variance (CV %) of AUC_{(0-∞)} compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg/0.1 ml to about 2 mg/0.1 ml and upon intranasal administration to human subjects provides at least about 10% reduction in coefficient of variance (CV %) of AUC_{(0-1 hr)} compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 0.8 mg/0.1 ml of dihydroergotamine and upon intranasal administration to human subjects provides at least about 10% reduction in coefficient of variance (CV %) of AUC_{(0-∞)} compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 0.65 mg/0.1 ml of dihydroergotamine and upon intranasal administration to human subjects provides at least about 10% reduction in coefficient of variance (CV %) of AUC_{(0-∞)} compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 0.6 mg/0.1 ml of dihydroergotamine and upon intranasal administration to human subjects provides at least about 10% reduction in coefficient of variance (CV %) of AUC_{(0-∞)} compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises about 0.55 mg/0.1 ml of dihydroergotamine and upon intranasal administration to human subjects provides at least about 10% reduction in coefficient of variance (CV %) of AUC_{(0-∞)} compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg to about 2 mg and upon intranasal administration to human subjects provide higher dC/dT value compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg/0.1 ml to about 2 mg/0.1 ml and upon intranasal administration to human subjects provides at least about a 10 percent higher dC/dT value compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg/0.1 ml to about 2 mg/0.1 ml and upon intranasal administration to human subjects provides at least about a 10 percent higher dC/dT value compared to a commercially-available 2 mg dihydroergotamine nasal dosage form, and said dC/dT value is measured in a single dose human pharmacokinetic study in a time period of Tomin to T_{15 mins}.

In another aspect of above embodiments, the present application relates to a pharmaceutical nasal dosage form comprising aqueous solution of dihydroergotamine or a pharmaceutically acceptable salt thereof, for treating migraine with or without aura in human subjects, wherein said dosage form comprises dihydroergotamine from about 0.5 mg/0.1 ml to about 2 mg/0.1 ml and upon intranasal administration to human subjects provides at least about a 10 percent higher dC/dT value compared to a commercially-available 2 mg dihydroergotamine nasal dosage form, and said dC/dT value is measured in a single dose human pharmacokinetic study in a time period of T_{0 min} to T_{15 mins}.

In another aspect of above embodiments, the present application relates to a pharmaceutical nasal dosage form comprising aqueous solution of dihydroergotamine or a pharmaceutically acceptable salt thereof, for treating migraine with or without aura in human subjects, wherein said dosage form comprises about 0.8 mg/0.lml of dihydroergotamine and upon intranasal administration to human subjects provides at least about a 10 percent higher dC/dT value compared to a commercially-available 2 mg dihydroergotamine nasal dosage form, and said dC/dT value is measured in a single dose human pharmacokinetic study in a time period of T_{0 min} to T_{15 mins}.

In another aspect of above embodiments, the present application relates to a pharmaceutical nasal dosage form comprising aqueous solution of dihydroergotamine or a pharmaceutically acceptable salt thereof, for treating migraine with or without aura in human subjects, wherein said dosage form comprises about 0.65 mg/0.1 ml of dihydroergotamine and upon intranasal administration to human subjects provides at least about a 10 percent higher dC/dT value compared to a commercially-available 2 mg dihydroergotamine nasal dosage form, and said dC/dT value is measured in a single dose human pharmacokinetic study in a time period of T_{0 min} to T_{15 mins}.

In another aspect of above embodiments, the present application relates to a pharmaceutical nasal dosage form comprising aqueous solution of dihydroergotamine or a pharmaceutically acceptable salt thereof, for treating migraine with or without aura in human subjects, wherein said dosage form comprises about 0.6 mg/0.1 ml of dihydroergotamine and upon intranasal administration to human subjects provides at least about a 10 percent higher dC/dT value compared to a commercially-available 2 mg dihydroergotamine nasal dosage form, and said dC/dT value is measured in a single dose human pharmacokinetic study in a time period of T_{0 min} to T_{15 mins}.

In another aspect of above embodiments, the present application relates to a pharmaceutical nasal dosage form comprising aqueous solution of dihydroergotamine or a pharmaceutically acceptable salt thereof, for treating migraine with or without aura in human subjects, wherein said dosage form comprises about 0.55 mg/0.1 ml of dihydroergotamine and upon intranasal administration to human subjects provides at least about a 10 percent higher dC/dT value compared to a commercially-available 2 mg dihydroergotamine nasal dosage form, and said dC/dT value is measured in a single dose human pharmacokinetic study in a time period of T_{0 min} to T_{15 mins}.

In another aspect of above embodiments, the present application relates to a pharmaceutical nasal dosage form comprising aqueous solution of dihydroergotamine or a pharmaceutically acceptable salt thereof, for treating migraine with or without aura in human subjects, wherein said dosage form comprises about 1.6 mg of dihydroergotamine and upon intranasal administration to human subjects provides at least about a 10 percent higher dC/dT value compared to a commercially-available 2 mg dihydroergotamine nasal dosage form, and said dC/dT value is measured in a single dose human pharmacokinetic study in a time period of T_{0 min} to T_{15 mins}.

In another aspect of above embodiments, the present application relates to a pharmaceutical nasal dosage form comprising aqueous solution of dihydroergotamine or a pharmaceutically acceptable salt thereof, for treating migraine with or without aura in human subjects, wherein said dosage form comprises about 1.3 mg of dihydroergotamine and upon intranasal administration to human subjects provides at least about a 10 percent higher dC/dT value compared to a commercially-available 2 mg dihydroergotamine nasal dosage form, and said dC/dT value is measured in a single dose human pharmacokinetic study in a time period of T_{0 min} to T_{15 mins}.

In another aspect of above embodiments, the present application relates to a pharmaceutical nasal dosage form comprising aqueous solution of dihydroergotamine or a pharmaceutically acceptable salt thereof, for treating migraine with or without aura in human subjects, wherein said dosage form comprises about 1.2 mg of dihydroergotamine and upon intranasal administration to human subjects provides at least about a 10 percent higher dC/dT value compared to a commercially-available 2 mg dihydroergotamine nasal dosage form, and said dC/dT value is measured in a single dose human pharmacokinetic study in a time period of T_{0 min} to T_{15 mins}.

In another aspect of above embodiments, the present application relates to a pharmaceutical nasal dosage form comprising aqueous solution of dihydroergotamine or a pharmaceutically acceptable salt thereof, for treating migraine with or without aura in human subjects, wherein said dosage form comprises about 1.1 mg of dihydroergotamine and upon intranasal administration to human subjects provides at least about a 10 percent higher dC/dT value compared to a commercially-available 2 mg dihydroergotamine nasal dosage form, and said dC/dT value is measured in a single dose human pharmacokinetic study in a time period of T_{0 min} to T_{15 mins}.

In another aspect of above embodiments, the present application relates to a pharmaceutical nasal dosage form comprising aqueous solution of dihydroergotamine or a pharmaceutically acceptable salt thereof, for treating migraine with or without aura in human subjects, wherein said dosage form comprises dihydroergotamine from about 0.5 mg/0.1 ml to about 2 mg/0.1 ml, one or more stabilizers in an amount of from about 0.01% w/w to about 10% w/w and at least one pharmaceutically acceptable excipient and upon intranasal administration to human subjects provides at least about a 10 percent higher dC/dT value compared to a commercially-available 2 mg dihydroergotamine nasal dosage form, and said dC/dT value is measured in a single dose human pharmacokinetic study in a time period of T_{0 min} to T_{15 mins}.

In another aspect of above embodiments, the present application relates to a pharmaceutical nasal dosage form comprising aqueous solution of dihydroergotamine or a pharmaceutically acceptable salt thereof, for treating migraine with or without aura in human subjects, wherein said dosage form comprises dihydroergotamine from about 0.5 mg/0.1 ml to about 2 mg/0.1 ml, one or more stabilizers in an amount of from about 0.01% w/w to about 5% w/w and at least one pharmaceutically acceptable excipient and upon intranasal administration to human subjects provides at least about a 10 percent higher dC/dT value compared to a commercially-available 2 mg dihydroergotamine nasal dosage form, and said dC/dT value is measured in a single dose human pharmacokinetic study in a time period of T_{0 min} to T_{15 mins}.

In another aspect of above embodiments, the present application relates to a pharmaceutical nasal dosage form comprising aqueous solution of dihydroergotamine or a pharmaceutically acceptable salt thereof, for treating migraine with or without aura in human subjects, wherein said dosage form comprises dihydroergotamine from about 0.5 mg/0.1 ml to about 2 mg/0.1 ml, one or more stabilizers in an amount of from about 0.01% w/w to about 2% w/w and at least one pharmaceutically acceptable excipient and upon intranasal administration to human subjects provides at least about a 10 percent higher dC/dT value compared to a commercially-available 2 mg dihydroergotamine nasal dosage form, and said dC/dT value is measured in a single dose human pharmacokinetic study in a time period of T_{0 min} to T_{15 mins}

In another aspect of above embodiments, the present application relates to a pharmaceutical nasal dosage form of dihydroergotamine for treating migraine with or without aura in human subjects, wherein said dosage form comprises dihydroergotamine from about 0.5 mg/0.1 ml to about 2 mg/0.1 ml and upon intranasal administration to human subjects provides from about 10 percent to about 30 percent higher dC/dT value compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg/0.1 ml to about 2 mg/0.1 ml and upon intranasal administration to human subjects provide at least about 20 percent higher dC/dT value compared to a commercially-available 2 mg dihydroergotamine nasal dosage form.

In another aspect of above embodiments, the present application relates to a pharmaceutical nasal dosage form of dihydroergotamine for treating migraine with or without aura in human subjects, wherein said dosage form comprises dihydroergotamine from about 0.5 mg/0.1 ml to about 2 mg/0.1 ml and upon intranasal administration to human subjects provide at least about 30 percent higher dC/dT value compared to a commercially-available 2 mg dihydroergotamine nasal dosage form.

In another aspect of above embodiments, the pharmaceutical nasal dosage form of dihydroergotamine of present application, upon intranasal administration to human subjects provide dC/dT value of at least about 1000(pg/mL)/hr in time period of 0 minutes to 15 minutes i.e. T_{0 min} to T_{15 mins}.

In another aspect of above embodiments, the present application relates to a method of administering pharmaceutical nasal dosage form of dihydroergotamine, wherein said administration requires less than about 15 minutes and less than four sprays to administer effective dose of dihydroergotamine for treating migraine with or without aura in human subj ects.

In another aspect of above embodiments, the administration of pharmaceutical nasal dosage form of dihydroergotamine comprises using suitable nasal device such as mono dose or bi-dose device for administering effective dose of dihydroergotamine for treating migraine with or without aura in human subjects.

In another aspect of above embodiments, the nasal devices used in the present application are pre-primed.

In another aspect of above embodiments, the present application relates to a method of administering pharmaceutical nasal dosage form of dihydroergotamine, wherein said dosage form is administered using pre-primed nasal devices and said administration requires less than about 15 minutes and less than four sprays to administer effective dose of dihydroergotamine.

In another aspect of above embodiments, the present application relates to a method of administering pharmaceutical nasal dosage form of dihydroergotamine, wherein said dosage form is administered using pre-primed nasal devices and said administration requires less than about 15 minutes and less than three sprays to administer effective dose of dihydroergotamine.

In another aspect of above embodiments, the present application relates to a method of administering pharmaceutical nasal dosage form of dihydroergotamine, wherein said dosage form is administered using pre-primed nasal devices and said administration requires less than about 15 minutes and less than two sprays to administer effective dose of dihydroergotamine.

In another aspect of above embodiments, the present application relates to a method of administering pharmaceutical nasal dosage form of dihydroergotamine, wherein said dosage form is administered using pre-primed nasal devices and said administration requires less than about 15 minutes and not more two sprays to administer effective dose of dihydroergotamine.

In another aspect of above embodiments, the present application relates to a method of administering pharmaceutical nasal dosage form of dihydroergotamine, wherein said dosage form is administered using pre-primed nasal devices and said administration requires less than about 15 minutes and at least one spray to administer effective dose of dihydroergotamine.

In another aspect of above embodiments, the present application relates to a method of administering pharmaceutical nasal dosage form of dihydroergotamine, wherein said dosage form is administered using pre-primed nasal devices and said dosage form comprises dihydroergotamine from about 0.5 mg/0.1 ml to about 2.0 mg/0.1 ml and at least one pharmaceutically acceptable excipient.

In another aspect of above embodiments, the present application relates to a method of administering pharmaceutical nasal dosage form of dihydroergotamine, wherein said dosage form is administered using pre-primed nasal devices and said dosage form comprises dihydroergotamine from about 0.5 mg/0.1 ml to about 2.0 mg/0.1 ml and at least one pharmaceutically acceptable excipient.

In another aspect of above embodiments, the method of administering pharmaceutical nasal dosage form of dihydroergotamine requires less than about 15 minutes and less than four sprays to administer effective dose of dihydroergotamine, wherein said effective dose is less than about 2 mg of dihydroergotamine.

In another aspect of the above embodiment, the method of administering pharmaceutical nasal dosage form of dihydroergotamine requires less than about 15 minutes and less than three sprays to administer effective dose of dihydroergotamine, wherein said effective dose is less than about 2 mg of dihydroergotamine.

In another aspect of the above embodiment, the method of administering pharmaceutical nasal dosage form of dihydroergotamine requires less than about 15 minutes and less than two sprays to administer effective dose of dihydroergotamine, wherein said effective dose is less than about 2 mg of dihydroergotamine.

In another aspect of the above embodiment, the method of administering pharmaceutical nasal dosage form of dihydroergotamine requires less than about 15 minutes and not more than two sprays to administer effective dose of dihydroergotamine, wherein said effective dose is less than about 2 mg of dihydroergotamine.

In another aspect of the above embodiment, the method of administering pharmaceutical nasal dosage form of dihydroergotamine requires less than about 15 minutes and at least one spray to administer effective dose of dihydroergotamine, wherein said effective dose is less than about 2 mg of dihydroergotamine.

In another aspect of above embodiments, the pre-primed nasal device can be a mono-dose device or bi-dose device, to administer effective dose of dihydroergotamine, wherein said device requires less than about 15 minutes for administration and said administration is into either one or both nostrils of the human subjects.

In another aspect of above embodiments, the present application relates to a method of administering pharmaceutical nasal dosage form of dihydroergotamine using pre-primed mono-dose nasal device, wherein said dosage form comprises dihydroergotamine from about 0.5 mg to about 2.0 mg and at least one pharmaceutically acceptable excipient, and said administration requires less than about 15 minutes to administer into either one or both nostrils of the human subjects.

In another aspect of above embodiments, the present application relates to a method of administering pharmaceutical nasal dosage form of dihydroergotamine using pre-primed mono-dose nasal device, wherein said dosage form comprises dihydroergotamine from about 0.5 mg/0.1 ml to about 2.0 mg/0.1 ml and at least one pharmaceutically acceptable excipient, and said administration requires less than about 15 minutes to administer into either one or both nostrils of the human subjects.

In another aspect of above embodiments, the present application relates to a method of administering pharmaceutical nasal dosage form of dihydroergotamine using pre-primed mono-dose nasal device, wherein said dosage form comprises dihydroergotamine from about 0.5 mg/0.1 ml to about 2.0 mg/0.1 ml and at least one pharmaceutically acceptable excipient, and said administration requires less than about 15 minutes to administer into one nostril of the human subjects.

In another aspect of above embodiments, the present application relates to a method of administering pharmaceutical nasal dosage form of dihydroergotamine using pre-primed mono-dose nasal device, wherein said dosage form comprises dihydroergotamine from about 0.5 mg/0.1 ml to about 2.0 mg/0.1 ml and at least one pharmaceutically acceptable excipient, and said administration requires less than about 15 minutes to administer into both the nostrils of the human subjects.

In some aspects of above embodiments, said pre-primed mono-dose nasal device has one nozzle or two nozzle.

In another aspect of above embodiments, the present application relates to a method of administering pharmaceutical nasal dosage form of dihydroergotamine using pre-primed mono-dose nasal device having one nozzle, wherein said dosage form comprises dihydroergotamine from about 0.5 mg/0.1 ml to about 2.0 mg/0.1 ml and at least one pharmaceutically acceptable excipient, and said administration requires less than about 15 minutes to administer into one nostril of the human subjects.

In another aspect of above embodiments, the present application relates to a method of administering pharmaceutical nasal dosage form of dihydroergotamine using pre-primed mono-dose nasal device having two nozzle, wherein said dosage form comprises dihydroergotamine from about 0.5 mg/0.1 ml to about 2.0 mg/0.1 ml and at least one pharmaceutically acceptable excipient, and said administration requires less than about 15 minutes to administer into both the nostrils of the human subjects.

In another aspect of above embodiments, the present application relates to a method of administering pharmaceutical nasal dosage form of dihydroergotamine using pre-primed bi-dose nasal device, wherein said dosage form comprises dihydroergotamine from about 0.5 mg to about 2.0 mg and at least one pharmaceutically acceptable excipient, and said administration requires less than about 15 minutes to administer into one or both nostrils of the human subjects.

In another aspect of above embodiments, the present application relates to a method of administering pharmaceutical nasal dosage form of dihydroergotamine using pre-primed bi-dose nasal device, wherein said dosage form comprises dihydroergotamine from about 0.5 mg/0.lml to about 2.0 mg/0.1 ml and at least one pharmaceutically acceptable excipient, and said administration requires less than about 15 minutes to administer into one or both nostrils of the human subjects.

In another aspect of above embodiments, the present application relates to a method of administering pharmaceutical nasal dosage form of dihydroergotamine using pre-primed bi-dose nasal device, wherein said dosage form comprises dihydroergotamine from about 0.5 mg/0.1 ml to about 2.0 mg/0.1 ml and at least one pharmaceutically acceptable excipient, and said administration requires less than about 15 minutes to administer into at least one nostril of the human subjects.

In another aspect of above embodiments, the present application relates to a method of administering pharmaceutical nasal dosage form of dihydroergotamine using pre-primed bi-dose nasal device, wherein said dosage form comprises dihydroergotamine from about 0.5 mg/0.1 ml to about 2.0 mg/0.1 ml and at least one pharmaceutically acceptable excipient, and said administration requires less than about 15 minutes to administer into both the nostrils of the human subjects.

In some aspects of above embodiments, said pre-primed bi-dose nasal device has one nozzle or two nozzle.

In another aspect of above embodiments, the present application relates to a method of administering pharmaceutical nasal dosage form of dihydroergotamine using pre-primed bi-dose nasal device having one nozzle, wherein said dosage form comprises dihydroergotamine from about 0.5 mg/0.1 ml to about 2.0 mg/0.1 ml and at least one pharmaceutically acceptable excipient, and said administration requires less than about 15 minutes to administer into at least one nostril of the human subjects.

In another aspect of above embodiments, the present application relates to a method of administering pharmaceutical nasal dosage form of dihydroergotamine using pre-primed bi-dose nasal device having two nozzle, wherein said dosage form comprises dihydroergotamine from about 0.5 mg/0.1 ml to about 2.0 mg/0.1 ml and at least one pharmaceutically acceptable excipient, and said administration requires less than about 15 minutes to administer into both the nostrils of the human subjects.

In another aspect of above embodiments, the method of administering pharmaceutical nasal dosage form of dihydroergotamine using pre-primed mono-dose or bi-dose nasal device having one or two nozzle, wherein said dosage form upon intranasal administration to human subjects provides higher AUC_{(0-15 mins)} compared to a commercially-available 2 mg dihydroergotamine nasal dosage form.

In another aspect of above embodiments, the method of administering pharmaceutical nasal dosage form of dihydroergotamine using pre-primed mono-dose or bi-dose nasal device having one or two nozzle, wherein said dosage form upon intranasal administration to human subjects provides at least one of the following pharmacokinetic parameters:
a. Cₘₐₓ of at least 900 pg/mL;
b. AUC₍₀₋ₜ₎ of at least 4500 pg*hr/mL; and
c. AUC_{(0-∞)} of at least 5000 pg*hr/mL.

In another aspect of above embodiments, the method of administering pharmaceutical nasal dosage form of dihydroergotamine using pre-primed mono-dose or bi-dose nasal device having one or two nozzle, wherein said dosage form upon intranasal administration to human subjects provides higher dC/dT value compared to a commercially-available 2 mg dihydroergotamine nasal dosage form.

In another aspect of above embodiments, the method of administering pharmaceutical nasal dosage form of dihydroergotamine using pre-primed mono-dose or bi-dose nasal device having one or two nozzle, wherein said dosage form upon intranasal administration to human subjects provides at least about a 10 percent higher dC/dT value compared to a commercially-available 2 mg dihydroergotamine nasal dosage form.

In another aspect of above embodiments, the method of administering pharmaceutical nasal dosage form of dihydroergotamine using pre-primed mono-dose or bi-dose nasal device having one or two nozzle, wherein said dosage form upon intranasal administration to human subjects provides at least about a 10 percent higher dC/dT value compared to a commercially-available 2 mg dihydroergotamine nasal dosage form, and said dC/dT value is measured in a single dose human pharmacokinetic study in a time period of T_{0 min} to

T15mins.

In another aspect of above embodiments, the method of administering pharmaceutical nasal dosage form of dihydroergotamine using pre-primed mono-dose or bi-dose nasal device having one or two nozzle, wherein said dosage form upon intranasal administration to human subjects provides dC/dT value of at least 1000 (pg/mL)/hr in time period of 0 minutes to 15 minutes.

In another aspect of above embodiments, the pre-primed nasal devices that can be used in the present application may be either a mono-dose nasal device or a bi-dose nasal device. These devices may be suitably assessed for its in-vitro spray performance by methods known in the art.

The in-vitro spray performance of these nasal devices may be characterized by the physical parameters such as, but not limited to, droplet size distribution, plume geometry and spray pattern. These physical parameters are determined by two-dimensional image analysis of the emitted plume using a non-impaction laser sheet-based instrument and an automated actuation station.

The droplet size distributions are expressed as D₁₀, D₅₀ and D₉₀. For example D₁₀ means 10% cumulative (from 0 to 100%) undersize droplet size distribution. In other words, if the droplet size D₁₀ is 7.8 μm, we can say 10% of the droplets in the tested sample are smaller than 7.8 μm, or the percentage of droplets smaller than 7.8 μm is 10%. Similarly D₅₀ and D₉₀ means 50% cumulative (from 0 to 100%) undersize droplet size distribution and 90% cumulative (from 0 to 100%) undersize droplet size distribution respectively.

Plume geometry is characterized by the spray angle and plume width. Spray angle is the angle of the emitted plume measured from the vertex of the spray cone and spray nozzle. Plume width is the width of the plume at a given distance (e.g. 3 cm) from the spray nozzle. The starting camera positions and software parameters were developed for the nasal spray, which is conventionally known in the art.

Spray pattern is characterized by the Dₘₐₓ (longest diameter), Dₘᵢₙ (shortest diameter), and Ovality Ratio (Dₘₐₓ/Dₘᵢₙ). Different frame rates, camera positions, and spray durations (start and stop time) were evaluated during method development. The frame rate was varied in order to select the optimal method with minimal background noise.

In another aspect of above embodiments, the present application relates to a pharmaceutical nasal dosage form of dihydroergotamine using pre-primed mono-dose or bi-dose nasal device, wherein said dosage form comprises dihydroergotamine from about 0.5 mg/0.1 ml to 2.0 mg/0.1 ml and at least one pharmaceutically acceptable excipient, and said device upon in-vitro spray performance characterization analysis provides at least one of the following droplet size parameters:
a. D₁₀: from about 10 μm to about 50 μm,
b. D₅₀: from about 10 μm to about 100 μm, and
c. D₉₀: from about 10 μm to about 300 μm.

In another aspect of above embodiments, the present application relates to a pharmaceutical nasal dosage form of dihydroergotamine using pre-primed mono-dose or bi-dose nasal device, wherein said dosage form comprises dihydroergotamine from about 0.5 mg/0.1 ml to 2.0 mg/0.1 ml and at least one pharmaceutically acceptable excipient, and said device upon in-vitro spray performance characterization analysis shows plume geometry (at 3 cm) of at least one of the following parameters:
a. Plume angle: from about 10° to about 120°,
b. Plume width: from about 0 mm to about 120 mm; and
c. Plume height: >85 mm.

In another aspect of above embodiments, the present application relates to a pharmaceutical nasal dosage form of dihydroergotamine administered using pre-primed mono-dose or bi-dose nasal device, wherein said dosage form comprises dihydroergotamine from about 0.5 mg/0.1 ml to about 2.0 mg/0.1 ml and at least one pharmaceutically acceptable excipient, and said device upon in-vitro spray performance characterization analysis shows spray pattern (at 3cm) of at least one of the following parameters:
a. Dmin: from about 10 mm to about 50 mm,
b. Dmax: from about 14 mm to about 90 mm and
c. Ovality: from about 0.0 to about 3.0.

In another aspect of above embodiments, the present application relates to pharmaceutical nasal dosage form of dihydroergotamine, wherein said dosage form comprises aqueous solution of dihydroergotamine from about 0.2 mg to about 2.0 mg in a volume of from about 0.05 ml to about 5.0 ml and at least one pharmaceutically acceptable excipient.

In another aspect of above embodiments, the present application relates to pharmaceutical nasal dosage form of dihydroergotamine, wherein said dosage form comprises aqueous solution of dihydroergotamine from about 0.2 mg to about 2.0 mg in a volume of from about 0.1 ml to about 1.0 ml and at least one pharmaceutically acceptable excipient.

In another aspect of above embodiments, the present application relates to pharmaceutical nasal dosage form of dihydroergotamine, wherein said dosage form comprises aqueous solution of dihydroergotamine at a concentration of from about 0.5 mg/0.1 ml to about 2.0 mg/0.1 ml and at least one pharmaceutically acceptable excipient.

In another aspect of above embodiments, the present application relates to pharmaceutical nasal dosage form of dihydroergotamine, wherein said dosage form comprises aqueous solution of dihydroergotamine at a concentration selected from the group of about 0.5 mg/0.1 ml or 0.55 mg/0.1 ml or 0.6 mg/0.1 ml or 0.65 mg/0.1 ml or about 0.7 mg/0.1 ml or about 0.8 mg/0.1 ml or about 0.9 mg/0.1 ml or about 1.0 mg/0.1 ml or about 1.1 mg/0.1 ml or about 1.2 mg/0.1 ml or about 1.3 mg/0.1 ml or about 1.4 mg/0.1 ml or about 1.5 mg/0.1 ml or about 1.6 mg/0.1 ml or about 1.7 mg/0.1 ml or about 1.8 mg/0.1 ml or about 1.9 mg/0.1 ml and about 2.0 mg/0.1 ml.

In another aspect of above embodiments, the present application relates to pharmaceutical nasal dosage form of dihydroergotamine, wherein said dosage form comprises aqueous solution of dihydroergotamine at a concentration of 0.8 mg/0.1 ml and at least one pharmaceutically acceptable excipient.

In another aspect of above embodiments, the present application relates to pharmaceutical nasal dosage form of dihydroergotamine, wherein said dosage form comprises aqueous solution of dihydroergotamine at a concentration of 0.65 mg/0.1 ml and at least one pharmaceutically acceptable excipient.

In another aspect of above embodiments, the present application relates to pharmaceutical nasal dosage form of dihydroergotamine, wherein said dosage form comprises aqueous solution of dihydroergotamine at a concentration of 0.6 mg/0.1 ml and at least one pharmaceutically acceptable excipient.

In another aspect of above embodiments, the present application relates to pharmaceutical nasal dosage form of dihydroergotamine, wherein said dosage form comprises aqueous solution of dihydroergotamine at a concentration of 0.55 mg/0.1 ml and at least one pharmaceutically acceptable excipient.

In another aspect of above embodiments, the pharmaceutical nasal dosage form of dihydroergotamine as disclosed herein does not show any precipitation upon storage for at least 7 days, at least 10 days, at least 15 days, at least 30 days, at least 45 days, at least 60 days or longer.

In another aspect of above embodiments, the pharmaceutical nasal dosage form of dihydroergotamine as disclosed herein does not show any precipitation upon storage such as at 2° C. to 8° C., 25° C., 40° C., or 45° C. for at least 7 days, at least 10 days, at least 15 days, at least 30 days, at least 45 days, at least 60 days or longer.

In another aspect of above embodiments, the pharmaceutical nasal dosage form of dihydroergotamine of present application may be prepared and filled in suitable nasal devices in sterile environment. The nasal dosage form is storage-stable for at least about 3 months at about 2° C. to 8° C., or 25° C. with about 60% relative humidity and or 40° C. with about 75% relative humidity.

In another aspect of above embodiments, the pharmaceutical nasal dosage form of dihydroergotamine of present application may be prepared and filled in suitable nasal devices in sterile environment. The nasal dosage form is storage-stable for at least about 6 months at about 2° C. to 8° C., or 25° C. with about 60% relative humidity and or 40° C. with about 75% relative humidity.

In another aspect of above embodiments, the pharmaceutical nasal dosage form of dihydroergotamine of present application may be prepared and filled in suitable nasal devices in sterile environment. The nasal dosage form is storage-stable for at least about 12 months at about 2° C. to 8° C., or 25° C. with about 60% relative humidity and or 40° C. with about 75% relative humidity.

In another aspect of above embodiments, the pharmaceutical nasal dosage form of dihydroergotamine of present application may be prepared and filled in suitable nasal devices in sterile environment. The nasal dosage form is storage-stable for at least about 18 months at about 2° C. to 8° C., or 25° C. with about 60% relative humidity and or 40° C. with about 75% relative humidity.

In another aspect of above embodiments, the pharmaceutical nasal dosage form of dihydroergotamine of present application may be prepared and filled in suitable nasal devices in sterile environment. The nasal dosage form is storage-stable for at least about 24 months at about 2° C. to 8° C., or 25° C. with about 60% relative humidity and or 40° C. with about 75% relative humidity.

In another aspect of above embodiments, the pharmaceutical nasal dosage form of dihydroergotamine of present application contains total impurities not more than about 5% or 4% or 3% or 2% or 1%.

In another aspect of above embodiments, the pharmaceutical nasal dosage form of dihydroergotamine of present application is an aqueous solution and having a pH of from about 2 to about 8, such as pH 2, pH 2.5, pH 3, pH 3.5, pH 4, pH 4.5, pH 5, pH 5.5, pH 6, pH 6.5, pH 7, pH 7.5 and pH 8.

In another aspect of above embodiments, the present application relates to a pharmaceutical nasal dosage form of dihydroergotamine for treating migraine with or without aura in human subjects, wherein said dosage form comprises inert gases such as carbon dioxide, nitrogen, helium, argon and the like.

In another aspect of above embodiments, the present application relates to a pharmaceutical nasal dosage form of dihydroergotamine for treating migraine with or without aura in human subjects, wherein said dosage form comprises nitrogen gas.

In another aspect of above embodiments, the aqueous solution of dihydroergotamine may be purged with inert gas and packaged in a suitable container comprising inert gas. The gas used for purging over the aqueous solution of dihydroergotamine and filled in the package may be same or different.

The final package may additionally contain pharmaceutically acceptable oxygen adsorbent. The container used for packaging or dispensing the nasal dosage form as per present application may be pouch, plastic container or container using suitable material known in the art.

The present application is further illustrated by the examples which are provided merely to be exemplary of the invention described above and do not limit the scope of the application. Certain modifications and equivalents will be apparent to those skilled in the art and are intended to be included within the scope of the present application.

### EXAMPLES

### Example 1-3

The compositions of dihydroergotamine mesylate (DHE) were prepared as listed below. The prepared compositions were evaluated for onset of precipitation.

TABLE 1

| | Ex. 1 | Ex. 2 | Ex. 3 |
|---|---|---|---|
| Ingredients | (% w/v) | | |
| Dihydroergotamine mesylate | 0.8 | 0.8 | 0.8 |
| Caffeine anhydrous | 1.5 | 1 | 2 |
| Dextrose anhydrous | 5.0 | 5.0 | 10.0 |
| Inert Gas (Nitrogen/Carbon | q.s. | | |
| dioxide/Helium, etc.) | | | |
| Water for Injection (WFI) | q.s. to 100% | | |
| Onset of Precipitation time | — | 1 day | 1 day |
| @ 45° C. | | | |

Manufacturing Process:

WFI was sparged with carbon dioxide, and caffeine, DHE and dextrose were dissolved to obtain a clear solution. The volume of solution was adjusted up to desired level followed by filtration. The filtered solution was stored in glass vials, closed with rubber stopper and aluminum seals.

### Example 4-5

The compositions of dihydroergotamine mesylate were prepared as listed below. The prepared compositions were evaluated for onset of precipitation.

TABLE 2

| | Ex. 4 | Ex. 5 |
|---|---|---|
| Ingredients | (% w/v) | |
| Dihydroergotamine mesylate | 0.8 | 0.8 |
| Glycerin | 15.0 | 60.0 |
| Ethanol | 7.5 | 30.0 |
| NaOH/Methane sulfonic acid | q.s. to | q.s. to |
| | pH 3.6 ± 0.2 | pH 3.6 ± 0.2 |
| Water for Injection (WFI) | q.s. to 100% | |
| Onset of Precipitation time | 1 day | No PPT till 19 days |
| @ 45° C. | | |

Manufacturing Process:

Sufficient quantity of methane sulfonic acid was added to WFI to achieve pH 3.6±0.2.

Ethanol, Glycerin and DHE were dissolved in WFI to obtain a clear solution. The volume of solution was adjusted up to desired level followed by filtration. The filtered solution was stored in glass vials, closed with rubber stopper and aluminum seals.

### Example 6-12

The compositions of dihydroergotamine mesylate were prepared comprising various stabilizing agents as listed below. The prepared compositions were evaluated for onset of precipitation.

TABLE 3

| | Ex. 6 | Ex. 7 | Ex. 8 | Ex. 9 | Ex. 10 | Ex. 11 | Ex. 12 |
|---|---|---|---|---|---|---|---|
| | (% w/v) | | | | | | |
| Dihydroergotamine | 0.8 | 0.8 | 0.8 | 0.8 | 0.8 | 0.8 | 0.8 |
| Mesylate | | | | | | | |
| Caffeine | 1.5 | 1.5 | 1.5 | 1.5 | 1.5 | 1.5 | 1.5 |
| Dextrose | 5.0 | 5.0 | 5.0 | 5.0 | 5.0 | 5.0 | 5.0 |
| Methane sulfonic | — | q.s. to | — | — | — | — | — |
| acid | | pH 4.0 | | | | | |
| Tri-sodium citrate | 0.15 | — | — | — | — | — | — |
| dihydrate | | | | | | | |
| Citric acid | 0.2 | — | 0.2 | — | — | — | — |
| monohydrate | | | | | | | |
| Ammonium acetate | — | — | — | 0.077 | — | — | — |
| Lysine acetate | — | — | — | — | 0.21 | — | — |
| Lysine HCl | — | — | — | — | — | 0.18 | — |
| Ascorbic acid | — | — | — | — | — | — | 0.1 |
| NaOH | — | — | — | — | — | — | q.s. to pH |
| | | | | | | | 4.2 |
| Acetic acid | — | — | — | q.s. to | q.s. to | — | — |
| | | | | pH 4.2 | pH 4.2 | | |
| HCl | — | — | — | — | — | q.s. to | — |
| | | | | | | pH 4.2 | |
| Water for injection | q.s. to 100% | | | | | | |
| Nitrogen | q.s | | | | | | |
| Observed pH | 3.99 | 3.98 | 2.61 | 4.25 | 4.17 | 4.22 | 4.18 |
| Observation of | 21 | 1 day | No | No | 2 days | 1 day | No PPT |
| Precipitation | days | | PPT | PPT | | | till 45 days |
| at 45° C. | | | till 40 | till 60 | | | |
| | | | days | days | | | |

Manufacturing Process:

WFI was sparged with nitrogen, and caffeine, DHE, and dextrose were dissolved to obtain a clear drug solution. This was followed by addition of stabilizing agents such as methane sulfonic acid, citric acid monohydrate, ammonium acetate, lysine acetate, lysine HC1 and ascorbic acid as shown in table above. The volume of solution was adjusted up to desired level. Formulations were filtered and solution was stored in glass vials, closed with rubber stopper and aluminum seals.

### Example 13-17

The compositions of dihydroergotamine mesylate (DHE) were prepared as listed below. The prepared compositions were evaluated for onset of precipitation.

TABLE 4

| Ingredients | Ex. 13 | Ex. 14 | Ex. 15 | Ex. 16 | Ex. 17 |
|---|---|---|---|---|---|
| Dihydroergotamine | 0.8 | 0.8 | 0.8 | 0.8 | 0.8 |
| Mesylate | | | | | |
| Caffeine | 1.5 | 1.5 | 1.5 | 1.5 | 1.5 |
| Dextrose | 5.0 | 5.0 | 5.0 | 5.0 | 5.0 |
| Tri-Sodium citrate | 0.2 | 0.2 | 0.2 | 0.2 | 0.2 |
| dihydrate | | | | | |
| Citric acid | 0.2 | 0.2 | 0.2 | 0.2 | 0.2 |
| monohydrate | | | | | |
| HPMC | 2.0 | — | — | — | — |
| Poloxamer 407 | — | 5.0 | — | — | — |
| Poloxamer 188 | — | — | 10.0 | — | — |
| Kollicoat IR | — | — | — | 5.0 | — |
| Kollidone VA64 | — | — | — | — | 5.0 |
| Water for injection | q.s. to 100% | | | | |
| Nitrogen | q.s. | | | | |
| Observation of | 17 days | No PPT till | 27 days | 3 days | 3 days |
| Precipitation | | 27 days | | | |
| at 45° C. | | | | | |

Manufacturing Process:

WFI was sparged with nitrogen, and caffeine, DHE, sodium citrate, citric acid and dextrose were dissolved to obtain a clear solution. Subsequently selected polymer such as HPMC/Poloxamer 407/Poloxamer 188/Kollicoat IR/Kollidone VA64 was dissolved. Finally, the volume of solution was adjusted up to desired level followed by filtration. The solution was stored in glass vials, closed with rubber stoppers and aluminum seals.

### Example 18 -23

The compositions of dihydroergotamine mesylate were prepared comprising citric acid and tri-sodium citrate dihydrate monohydrate. The prepared compositions were evaluated for onset of precipitation.

TABLE 5

| | Ex. 18 | Ex. 19 | Ex. 20 | Ex. 21 | Ex. 22 | Ex. 23 |
|---|---|---|---|---|---|---|
| Ingredient | (% w/v) | | | | | |
| Dihydroergotamine | 0.8 | 0.8 | 0.8 | 0.8 | 0.8 | 0.8 |
| mesylate | | | | | | |
| Caffeine anhydrous | 1.5 | 1.5 | 1.5 | 1.5 | 1.5 | 1.5 |
| Dextrose anhydrous | 5.0 | 5.0 | 5.0 | 5.0 | 5.0 | 5.0 |
| Citric acid monohydrate | 0.20 | 0.21 | 0.20 | 0.21 | 0.20 | 0.00 |
| Tri-Sodium citrate dehydrate | 0.20 | 0.18 | 0.15 | 0.12 | 0.00 | 0.00 |
| PH | 4.2 | 4.0 | 3.8 | 3.6 | 2.6 | 4.65 |
| Inert Gas | q.s | | | | | |
| Water for Injection | q.s to 100% | | | | | |
| (WFI) | | | | | | |
| No. of days without | 13 | 14 | 20 | 25 | 60 | 2 |
| precipitation @ 45° C. | | | | | | |

Precipitation was not observed after 60 days. The study was discontinued thereafter

Procedure:

Manufacturing Process:

WFI was sparged with nitrogen, and caffeine, DHE, and dextrose were dissolved to obtain a clear drug solution. Subsequently required quantity of citric acid monohydrate and tri-Sodium citrate dihydrate was dissolved. Finally, the volume of solution was adjusted up to desired level followed by filtration. The solution was stored in glass vials, closed with rubber stopper and aluminum seals.

### Example 24-25

The compositions of dihydroergotamine mesylate were prepared comprising various co-solvents as listed below. The prepared compositions were evaluated for onset of precipitation.

TABLE 6

| Material | Ex. 24 | Ex. 25 |
|---|---|---|
| Ingredients | (% w/v) | |
| Dihydroergotamine Mesylate | 0.8 | 0.8 |
| Caffeine | 1.5 | 1.5 |
| Dextrose | 5.0 | 5.0 |
| Tri-Sodium citrate dihydrate | 0.2 | — |
| Citric acid monohydrate | 0.2 | — |
| Ethanol | 2.0 | — |
| Methoxy polyethylene glycol (m-PEG) | — | 6.0 |
| Water for injection | q.s. to 100% | q.s. to 100% |
| Nitrogen | q.s. | q.s. |
| Observed pH | 4.13 | 4.48 |
| Onset of Precipitation at 45° C. | 10 days | 3 days |

Manufacturing Process:

WFI was sparged with nitrogen, and caffeine, DHE and dextrose were dissolved to obtain a clear solution. In Ex. 24 sodium citrate, citric acid and ethanol were added to obtain a clear solution with acidic pH. Whereas in Ex. 25, only m-PEG was added to clear solution with acidic pH. The volume of solution was adjusted to desired level followed by filtration. The solution was stored in glass vials, closed with rubber stopper and aluminum seals.

### Example 26

The compositions of examples were further evaluated for stability testing at 2-8° C., 25° C./60% RH and 40° C./75% RH. The observations are listed in table no.

TABLE 7

| | Stability | Time | | Assay | SHUI* | Impurity D | |
|---|---|---|---|---|---|---|---|
| | Condition | point | Precipitation | (%) | (%) | (%) | Total Impurity % |
| Ex. 06 | 2-8° C. | 6 M | No | 102.10 | 0.10 | 0.15 | 0.65 |
| Ex. 06 | 25° C./60% RH | 6 M | No | 99.60 | 0.36 | 1.29 | 2.65 |
| Ex. 06 | 40° C./75% RH | 3 M | No | 89.97 | 0.59 | 5.22 | 7.79 |
| Ex. 09 | 25° C./60% RH | 3 M | No | 99.70 | 0.14 | 0.26 | 1.14 |
| Ex. 09 | 40° C./75% RH | 3 M | No | 98.50 | 0.30 | 1.87 | 3.11 |
| Ex. 09 | 25° C./60% RH | 6 M | No | 99.70 | 0.11 | 0.32 | 0.96 |
| Ex. 12 | 25° C./60% RH | 3 M | No | 97.30 | 0.07 | 0.12 | 0.47 |
| Ex. 12 | 40° C./75% RH | 3 M | No | 95.50 | 0.26 | 0.95 | 1.63 |

SHUI—Single Highest unknown Impurity

### Example 27-33

The compositions of dihydroergotamine mesylate were prepared comprising various surfactants such as benzalkonium chloride, DDM, Vitamin E TPGS, Polysorbate 80. The prepared compositions were evaluated for onset of precipitation.

TABLE 8

| | Ex. 27 | Ex. 28 | Ex. 29 | Ex. 30 | Ex. 31 | Ex. 32 | Ex.33 |
|---|---|---|---|---|---|---|---|
| Ingredients | (% w/v) | | | | | | |
| Dihydroergotamine | 0.8 | 0.8 | 0.4 | 0.4 | 0.8 | 0.8 | 0.8 |
| Mesylate | | | | | | | |
| Caffeine | 1.5 | 1.5 | 1.0 | 1.5 | 1.5 | 1.5 | 1.5 |
| Dextrose | 5.0 | 5.0 | 5.0 | 5.0 | 5.0 | 5.0 | 5.0 |
| Tri-Sodium citrate | — | 0.2 | 0.2 | 0.2 | — | 0.2 | 0.2 |
| dehydrate | | | | | | | |
| Citric acid | — | 0.2 | 0.2 | 0.2 | — | 0.2 | 0.2 |
| monohydrate | | | | | | | |
| Benzalkonium | 0.5 | 0.12 | — | — | — | — | — |
| chloride | | | | | | | |
| DDM | — | — | 0.1 | 0.2 | — | — | — |
| Vitamin E TPGS | — | — | — | — | 1.0 | 0.2 | |
| Polysorbate 80 | — | — | — | — | — | — | 1.0 |
| Water for injection | q.s. to 1.0 mL | | | | | | |
| Nitrogen | q.s. | | | | | | |
| Onset of | No PPT till | 12 days | No | 9 days | No | 21 | No |
| Precipitation at | 20 days | | PPT | | PPT | days | PPT |
| 45° C. | | | till 27 | | till 60 | | till 13 |
| | | | days | | days | | days |

Manufacturing Process:

WFI was sparged with nitrogen, and caffeine, DHE, and dextrose were dissolved to obtain a clear drug solution. Stabilizing agents such as sodium citrate and citric acid were dissolved in respective examples as mentioned in table shown above. Subsequently required quantity of surfactant benzalkonium chloride/DDM/Vitamin E TPGS/Polysorbate 80 was dissolved. Finally, the volume of solution was adjusted up to desired level followed by filtration. The solution was stored in glass vials, closed with rubber stopper and aluminum seals.

### Example 34-37

The compositions of dihydroergotamine mesylate were prepared comprising vitamin E TPGS and evaluated for onset of precipitation.

TABLE 9

| | Ex. 34 | Ex. 35 | Ex. 36 | Ex. 37 |
|---|---|---|---|---|
| Ingredients | (% w/v) of ingredients | | | |
| Dihydroergotamine mesylate | 0.8 | 0.8 | 0.8 | 0.8 |
| Caffeine anhydrous | 1.5 | 1.5 | 1.5 | 1.5 |
| Dextrose anhydrous | 5.0 | 5.0 | 5.0 | 5.0 |
| Vitamin E TPGS | 1 | 0.75 | 0.5 | 0.2 |
| Inert Gas | q.s | | | |
| Water for Injection (WFI) | q.s to 100% | | | |
| No. of days without precipitation | 60 | 8 | 1 | 0 |
| @ 45° C. | | | | |

Precipitation was not observed after 60 days. The study was discontinued thereafter.

Manufacturing Process:

WFI was sparged with nitrogen, and caffeine, DHE, and dextrose were dissolved to obtain a clear drug solution. Subsequently required quantity of Vitamin E TPGS was dissolved. Finally, the volume of solution was adjusted up to desired level followed by filtration. The solution was stored in glass vials, closed with rubber stopper and aluminum seals.

### Example 38-40

The compositions of dihydroergotamine mesylate were prepared comprising vitamin E TPGS, citric acid and tri-sodium citrate dihydrate monohydrate. The prepared compositions were evaluated for onset of precipitation.

TABLE 10

| | Ex. 38 | Ex. 39 | Ex. 40 |
|---|---|---|---|
| Ingredient | (% w/v) of ingredients | | |
| Dihydroergotamine mesylate | 0.8 | 0.8 | 0.8 |
| Caffeine anhydrous | 1.5 | 1.5 | 1.5 |
| Dextrose anhydrous | 5.0 | 5.0 | 5.0 |
| Citric acid monohydrate | 0.20 | 0.20 | 0.20 |
| Tri-Sodium citrate dihydrate | 0.20 | 0.20 | 0.20 |
| Vitamin E TPGS | 0.75 | 0.5 | 0.2 |
| pH | 4.2 | 4.2 | 4.2 |
| Inert Gas | q.s. | | |
| Water for Injection (WFI) | q.s to 100% | | |
| No. of days without precipitation @ 45° C. | 21 | 19 | 15 |

Manufacturing Process:

WFI was sparged with nitrogen, and caffeine, DHE, and dextrose were dissolved to obtain a clear drug solution. Subsequently required quantity of citric acid monohydrate, tri-Sodium citrate dihydrate and Vitamin E TPGS was dissolved. Finally, the volume of solution was adjusted up to desired level followed by filtration. The solution was stored in glass vials, closed with rubber stopper and aluminum seals.

### Example 41.

The compositions of examples as listed below were further evaluated for stability testing at 2-8° C., 25° C./60% RH and 40° C./75% RH. The observations are listed in table no.

TABLE 11

| | Stability | Time | | Assay | SHUI* | Impurity D | % Total |
|---|---|---|---|---|---|---|---|
| | Condition | point | Precipitation | (%) | (%) | (%) | Impurity |
| Ex. 27 | 2-8° C. | 6 M | No | 101.3 | | 0.07 | 0.64 |
| Ex. 27 | 25° C./60% RH | 6 M | No | 100 | 0.11 | 0.71 | 1.35 |
| Ex. 27 | 40° C./75% RH | 6 M | No | 92.2 | 0.21 | 7.63 | 8.65 |
| Ex. 28 | 2-8° C. | 6 M | No | — | 0.10 | 0.07 | 0.62 |
| Ex. 28 | 25° C./60% RH | 6 M | No | — | 0.09 | 0.78 | 1.36 |
| Ex. 28 | 40° C./75% RH | 6 M | No | — | 0.53 | 6.21 | 8.11 |
| Ex. 31 | 2-8° C. | 6 M | No | 102.60 | 0.10 | 0.06 | 0.52 |
| Ex. 31 | 25° C./60% RH | 6 M | No | 100.70 | 0.13 | 0.33 | 1.09 |
| Ex. 31 | 40° C./75% RH | 6 M | No | 95 | 0.37 | 4.46 | 6.14 |

SHUI—Single Highest unknown Impurity

### Example 42

Physical characteristics of spray emitted from nasal device comprising compositions of Example no 20 were determined.

The droplet size distribution of spray produced by nasal devices was measured by Malvern Spraytec apparatus. 11 units were actuated to measure droplet size using Malvern Spraytec apparatus. 1st unit was used for trial actuation and remaining 10 units were used for data generation and analysis. The following parameters were used for droplet size distribution using Malvern Spraytec apparatus. The observations are shown below.

| | |
|---|---|
| Instrument setting | Input parameter |

Spray VIEW NSx Actuation station

| Trigger source | External | |
|---|---|---|
| Profile | Symmetric | |
| Characterization | Manual | |
| Shot count | 1 (for each actuation) | |
| 1^{st} stroke length | 16.2 | mm |
| 2^{nd} stroke length | 9.8 | mm |
| Contact force | 0.3 | kg |
| Velocity | 70 | mm/s |
| Acceleration | 5000 | mm/s² |
| Initial Delay | 30 | ms |
| Hold Time | 300 | ms |
| Final delay | 0 | ms |

Malvern SprayTec Software

| Vertical distance from the laser beam | 30 | mm |
|---|---|---|
| Measurement type | Rapid | |
| Lens type | 300 | mm |
| Angle | 0° | |
| Data acquisition rate | 500 | Hz |
| Background duration | 10 | sec |
| Transmission | 90 | |
| Number of events | 2 | |
| Duration per event | 150 | msec |
| Data collection start before the trigger | 50 | msec |

| | |
|---|---|
| Multiple scattering analysis | Disable |
| Stable phase selection | Manual |

TABLE 12

| | Droplet size distribution (Average value, n = 10) | |
|---|---|---|
| | Example 20 | |
| Parameters | Spray 1 | Spray 2 |
| D₁₀ (μm) | 19.88 | 19.17 |
| D₅₀ (μm) | 37.94 | 36.44 |
| D₉₀ (μm) | 79.44 | 75.32 |
| % V < 10 μm (%) | 0.01 | 0.01 |
| Span | 1.57 | 1.54 |

TABLE 13

| | Droplet size distribution (Average value, n = 10) | |
|---|---|---|
| | Example 34 | |
| Parameters | Spray 1 | Spray 2 |
| D₁₀ (μm) | 19.77 | 19.34 |
| D₅₀ (μm) | 37.36 | 36.09 |
| D₉₀ (μm) | 73.62 | 69.94 |
| % V < 10 μm (%) | 0.00 | 0.00 |
| Span | 1.44 | 1.40 |

The spray pattern and plume geometry analysis as measured by a laser sheet based analysis instrument, “SprayVIEW NSP (Proveris Scientific, US)”. Units were actuated using a SprayVIEW Automated Actuation System., twenty units were actuated in the SprayVIEW NSP. 11 units were tested for spray pattern of which 1^{st} unit was used for trail actuation and other 10 units were used for data generation and analysis. 11 units were tested for plume geometry of which Pt unit was used for trail actuation and other 10 units were used for data generation and analysis. The observations are shown below.

For spray pattern analysis:

The following parameters were used for spray pattern analysis using SprayVIEW NSP:

| | |
|---|---|
| Instrument setting | Input parameter |

Spray VIEW NSx Actuation station

| Characterization | Manual | |
|---|---|---|
| Profile | Symmetric | |
| 1^{st} stroke length | 16.2 | mm |
| 2^{nd} stroke length | 9.8 | mm |
| Contact force | 0.3 | kg |
| Velocity | 70 | mm/s |
| Acceleration | 5000 | mm/s² |
| Initial Delay | 30 | ms |
| Hold Time | 300 | ms |
| Final delay | 0 | ms |

Spray VIEW NSP

| Orifice Tip distance | 30 | mm |
|---|---|---|
| Shot count | 1 (for each actuation) | |
| Frame Rate | 200 | HZ |
| Number if images to acquire | 100 | |
| Lens Aperture | 2.0 | |
| Camera Position Horizontal (From Right) | 7 | cm |
| Camera Height (top of truck) | 33 | cm |
| Laser Position (Left of truck) | 8.6 | cm |
| Laser Position (from right) | 5.5 | cm |

| | |
|---|---|
| Threshold | 6 |
| palette | Gradient |

For Plume Geometry Analysis:

The following parameters were used for plume geometry analysis using SprayVIEW NSP:

| | |
|---|---|
| Instrument setting | Input parameter |

Spray VIEW NSx Actuation station

| Characterization | Manual | |
|---|---|---|
| Profile | Symmetric | |
| 1^{st} stroke length | 16.2 | mm |
| 2^{nd} stroke length | 9.8 | mm |
| Contact force | 0.3 | kg |
| Velocity | 70 | mm/s |
| Acceleration | 5000 | mm/s² |
| Initial Delay | 30 | ms |
| Hold Time | 300 | ms |
| Final delay | 0 | ms |

Spray VIEW NSP

| Plume distance | 3 | cm |
|---|---|---|
| Shot count | 1 (for each actuation) | |
| Frame Rate | 200 | HZ |
| Number if images to acquire | 100 | |
| Lens Aperture | 2.0 | |
| Camera Position (From Right) | 33 | cm |
| Camera Height (top of truck) | 9 | cm |
| Laser Positon (Left of truck) | 8.6 | cm |
| Laser depth (from right) | 5.5 | cm |
| Lased Height (Top of truck) | 14.5 | cm |
| Plume orientation | 0° | l |

| | |
|---|---|
| Time delay (Frame) | Select from plateau region |
| | and record (Snapshot) |
| Arm 1&2 (%) | Analyst select manually and report |
| palette | Gradient |

TABLE 14

Spray Pattern (Average value, n = 10)
Example 20

| Parameters | Spray 1 | Spray 2 |
|---|---|---|
| Dmin (mm) | 26.02 | 29.16 |
| Dmax (mm) | 33.87 | 35.87 |
| Ovality Ratio | 1.30 | 1.23 |

TABLE 15

| | |
|---|---|
| | Plume Geometry |
| | (Average value, n = 10) |
| Batch no. | Example 20 |

| Parameters | Spray 1 | Spray 2 |
|---|---|---|
| Plume Angle (°) | 67.87 | 68.6 |
| Plume Width (mm) | 40.508 | 41.092 |

TABLE 16

Spray Pattern
(Average value, n = 10)
Example 34

| Parameters | Spray 1 | Spray 2 |
|---|---|---|
| Dmin (mm) | 30.37 | 31.36 |
| Dmax (mm) | 36.51 | 37.63 |
| Ovality Ratio | 1.21 | 1.20 |

TABLE 17

| | |
|---|---|
| | Plume Geometry |
| | (Average value, n = 10) |
| Batch no. | Example 34 |

| Parameters | Spray 1 | Spray 2 |
|---|---|---|
| Plume Angle (°) | 67.11 | 67.15 |
| Plume Width (mm) | 39.976 | 39.935 |

### Example 43

An open label, single-dose, two-treatment, comparative bioavailability study of Example 1 versus MIGRANAL® nasal spray (0.5 MG/INH) in 18 healthy adult male subjects under fasting conditions were conducted.

Method:

Treatment 1:

Subjects were administered 0.1 ml (containing 0.8 mg of dihydroergotamine mesylate) of Example 1 in each nostril, for a total dosage of 1.6 mg of dihydroergotamine mesylate, in two sprays.

Treatment 2:

Subjects were administered MIGRANAL® nasal spray as per its monograph or label. One spray (0.5 mg of dihydroergotamine mesylate) of MIGRANAL® was administered in each nostril. Fifteen minutes later, an additional one spray (0.5 mg of dihydroergotamine mesylate) of MIGRANAL® was administered in each nostril, for a total dosage of 2.0 mg of dihydroergotamine mesylate, in four sprays.

Results:

The pharmacokinetic parameters that were observed are listed in Table given below:

TABLE 18

| PK Parameters* | Example 1 | Migranal |
|---|---|---|
| Cₘₐₓ | 728 | 800 |
| (pg/mL) | | |
| Tₘₐₓ | 0.75 | 0.75 |
| (hr) | | |
| AUC₍₀₋ₜ₎ | 3466 | 3462 |
| (pg * hr/mL) | | |
| AUC_{(0-∞)} | 3923 | 3910 |
| (pg * hr/mL) | | |
| AUC_{(0-5 min)} | 2.8 | 0.6 |
| (pg * hr/mL) | | |
| AUC_{(0-15 min)} | 33 | 17 |
| (pg * hr/mL) | | |
| AUC_{(0-30 min)} | 148 | 113 |
| (pg * hr/mL) | | |
| AUC_{(0-1 hr)} | 464 | 464 |
| (pg * hr/mL) | | |
| dC/dT_{(0-15 mins)} | 1226.4 | 704 |
| (pg/mL)/hr | | |

All the values provided above are in mean value except Tₘₐₓ which is median.

### Example 44

The pharmacokinetic parameters of Example 20 is extracted from an open-label, randomized, single dose, three-treatment, three-period, six sequence, crossover comparative bioavailability study of test formulations versus MIGRANAL® nasal spray (0.5 MG/INH) in 18 healthy adult male subjects under fasting conditions were conducted.

Method:

Treatment A with Example No 20:

Subjects were administered 0.1 ml (containing 0.8 mg of dihydroergotamine mesylate) of Example 20 in each nostril, for a total dosage of 1.6 mg of dihydroergotamine mesylate, in two sprays.

Treatment with Migranal:

Subjects were administered MIGRANAL® nasal spray as per its monograph or label. One spray (0.5 mg of dihydroergotamine mesylate) of MIGRANAL® was administered in each nostril. Fifteen minutes later, an additional one spray (0.5 mg of dihydroergotamine mesylate) of MIGRANAL® was administered in each nostril, for a total dosage of 2.0 mg of dihydroergotamine mesylate, in four sprays.

Results:

The pharmacokinetic parameters that were observed are listed in Table given below:

TABLE 19

| | PK Parameters* | Example 20 | Migranal ® |
|---|---|---|---|
| Geo LSM | Cₘₐₓ | 1157 | 776 |
| | (pg/mL) | | |
| | AUC₍₀₋ₜ₎ | 5628 | 3962 |
| | (pg * hr/mL) | | |
| | AUC (0-∞) | 6270 | 4432 |
| | (pg * hr/mL) | | |
| Mean | AUC_{(0-Ref tmax)} | 456 | 309 |
| | (pg * hr/mL) | | |
| Median value | Tmax (hrs) | 0.75 | 0.88 |

TABLE 20

| | | Treatment A | Treatment C |
|---|---|---|---|
| Sr. No | PK parameters | Citrate buffer | Migranal ® |
| 1 | dC/dT_{(0-15 mins)} | 1354 | 927 |

TABLE 21

| | | | | % Reduction |
|---|---|---|---|---|
| | | | | in time in |
| | | Treatment A | | comparison |
| | | Citrate | Treatment C | Treatment C |
| Sr. No | PK parameters | buffer | Migranal ® | Migranal ® |
| 1 | Time to reach | Less than 30 | 45 minutes | 33.33% |
| | Cₘₐₓ of Migranal ® | minutes | | |

TABLE 22

| | | | | % Reduction in |
|---|---|---|---|---|
| | PK parameters | Treatment A | | comparison to |
| | (% Coefficient of | Citrate | Treatment C | Treatment C |
| Sr. No | variation) | buffer | Migranal ® | Migranal ® |
| 1 | Cₘₐₓ | 34 | 50 | 32 |
| 2 | AUC_{(0-2 hr)} | 35 | 47 | 26 |
| 3 | AUC₍₀₋ₜ₎ | 31 | 45 | 31 |
| 4 | AUC_{(0-inf)} | 29 | 43 | 33 |

### Example 45

The pharmacokinetic parameters of Example 34 is extracted from an open-label, randomized, single dose, three-treatment, three-period, six sequence, crossover comparative bioavailability study of test formulations versus MIGRANAL® nasal spray (0.5 MG/INH) in 18 healthy adult male subjects under fasting conditions were conducted.

Method:

Treatment B with Example No 34:

Subjects were administered 0.1 ml (containing 0.8 mg of dihydroergotamine mesylate) of Example 13 in each nostril, for a total dosage of 1.6 mg of dihydroergotamine mesylate, in two sprays.

Treatment with Migranal:

Subjects were administered MIGRANAL® nasal spray as per its monograph or label. One spray (0.5 mg of dihydroergotamine mesylate) of MIGRANAL® was administered in each nostril. Fifteen minutes later, an additional one spray (0.5 mg of dihydroergotamine mesylate) of MIGRANAL® was administered in each nostril, for a total dosage of 2.0 mg of dihydroergotamine mesylate, in four sprays.

Results:

The pharmacokinetic parameters that were observed are listed in Table given below:

TABLE 23

| | PK Parameters* | Example 34 | Migranal ® |
|---|---|---|---|
| Geo LSM | Cₘₐₓ | 973 | 776 |
| | (pg/mL) | | |
| | AUC₍₀₋ₜ₎ | 4833 | 3962 |
| | (pg * hr/mL) | | |
| | AUC (0-∞) | 5400 | 4432 |
| | (pg * hr/mL) | | |
| Mean | AUC_{(0-Ref tmax)} | 395 | 309 |
| | (pg * hr/mL) | | |
| Median value | Tmax (hrs) | 0.75 | 0.88 |

TABLE 24

| | | | Treatment C |
|---|---|---|---|
| Sr. No | PK parameters | Example 34 | Migranal ® |
| 1 | dC/dT_{(0-15 mins)} | 1461 | 927 |

TABLE 25

| | | | | % Reduction |
|---|---|---|---|---|
| | | | | in time in |
| | | | | comparison |
| | | | Treatment C | Treatment C |
| Sr. No | PK parameters | Example 34 | Migranal ® | Migranal ® |
| 1 | Time to reach | Less than 30 | 45 minutes | 33.33% |
| | | minutes | | |
| | C_{max of} | | | |
| | Migranal ® | | | |

TABLE 26

| | | | | % Reduction in |
|---|---|---|---|---|
| | PK parameters | | | comparison to |
| | (% Coefficient | | Treatment C | Treatment C |
| Sr. No | of variation) | Example 34 | Migranal ® | Migranal ® |
| 1 | Cₘₐₓ | 25 | 50 | 50 |
| 2 | AUC_{(0-2 hr)} | 24 | 47 | 48.9 |
| 3 | AUC₍₀₋ₜ₎ | 32 | 45 | 28.9 |
| 4 | AUC_{(0-inf)} | 29 | 43 | 32.6 |

### Example 46 & 47

TABLE 27

| | Ex. 46 | |
|---|---|---|
| Ingredient | mg/ml | Ex. 47 |
| Dihydroergotamine | 6 | 6 |
| mesylate | | |
| Caffeine anhydrous | 15 | 15 |
| Dextrose anhydrous | 50 | 50 |
| Ammonium acetate | 0.7708 | — |
| Acetic Acid | q.s to pH 4.2 | — |
| Ascorbic Acid | — | 1 |
| Sodium Hydroxide | — | q.s to pH 4.2 |
| Water for injection | q.s to 1 ml | q.s to 1 ml |

Manufacturing Process:

WFI was sparged with nitrogen, and caffeine, DHE, and dextrose were dissolved to obtain a clear drug solution. This was followed by addition of stabilizing agents such as ammonium acetate and ascorbic acid as shown in table above. The volume of solution was adjusted up to desired level. Formulations were filtered and solution was stored in glass vials, closed with rubber stopper and aluminum seals.

### Example 48

The pharmacokinetics of compositions of Example no 46, 47 and Migranal® were studied in rat models.

The rats were administered compositions of Examples 46, 47 and Migranal® as shown in table 27 and blood samples was collected to determine pharmacokinetic parameters.

Method:

Treatments:

Each group consists of 6 rats.
Group 1: Composition of Example no 46 were dosed @ 12.5 μL/each nostril once.
Group 2: Composition of Example no 47 were dosed @ 12.5 μL/each nostril once.
Group 3: Migranal® was dosed @ 12.5 μL/each nostril twice with 15 min interval between each dosing.

Time points for blood collection:
Points: 2 mins, 5 mins, 15 mins (pre-dose for second dose of Migranal®), 20 mins, 30 mins; and 1, 2, 4, 8, & 24 hr

TABLE 28

| | | Dose | Strength | Dose Volume |
|---|---|---|---|---|
| Group | | (mg/rat) | (mg/mL) | (μL/rat) |
| 1 | Ex. 46 | 0.15 | 6 | 25 |
| 2 | Ex. 47 | 0.15 | 6 | 25 |
| 3 | Migranal ® | 0.2 | 4 | 50 |

Results:

The pharmacokinetic parameters that were observed are listed in Table no 16 given below

TABLE 29

| | Ex. 46 | Ex. 47 | Migranal ® |
|---|---|---|---|
| Strength (mg/mL) | 6 | 6 | 4 |
| Dose (mg/rat) | 0.15 | 0.15 | 0.2 |
| Dosing | Once to | Once to | Twice to each |
| | each nostril | each nostril | nostril |
| Median tₘₐₓ (min) | 15 (15-30) | 25 (15-60) | 30 (20-60) |
| Cₘₐₓ (ng/mL) | 12 | 21 | 20 |
| *DN_Cₘₐₓ (ng/mL/mg) | 77 | 141 | 102 |
| AUC_{(0-1 hr)} (hr * ng/mL) | 8 | 14 | 12 |
| AUC_{(0-2 hr)} (hr * ng/mL) | 14 | 23 | 23 |
| AUCₗₐₛₜ (hr * ng/mL) | 30 | 43 | 56 |
| AUC_{Inf} (hr * ng/mL) | 34 | 47 | 59 |
| *DN_AUC_{Inf} | 225 | 315 | 294 |
| T_{1/2} (hr) | 2.6 | 2.8 | 3.7 |
| F % | 77 | 107 | 100 |

DN = Data of dose normalized of Ex. 46 & 47 to Migranal ®

1. A pharmaceutical nasal dosage form, comprising: dihydroergotamine or a pharmaceutically acceptable salt thereof and at least one pharmaceutically acceptable excipient for treating migraine with or without aura in human subjects, wherein said dosage form is provided in a pre-primed nasal device and said dosage form requires less than about 15 minutes to administer an effective dose of dihydroergotamine.

2. The nasal dosage form of claim 1, wherein said dosage form provided in the pre-primed nasal device requires less than four sprays to administer said effective dose of dihy droergotamine.

3. The nasal dosage form of claim 1, wherein said effective dose is from about 0.5 mg to about 2.0 mg.

4. The nasal dosage form of claim 1, wherein said dosage form further comprises one or more stabilizers.

5. The nasal dosage form of claim 4, wherein said stabilizers are present in an amount of from about 0.01% w/w to about 10% w/w.

6. The nasal dosage form of claim 4, wherein said stabilizers are selected from the group of stabilizers consisting of: citric acid, tartaric acid, ascorbic acid, acetic acid, formic acid, methanoic acid, fumaric acid, propionic acid, butanoic acid, ethanoic acid, benzoic acid, butyric acid, malic acid, propionic acid, epoxysuccinic acid, muconic acid, furanacrylic acid, citramalic acid, capric acid, stearic acid, caproic acid, malonic acid, succinic acid, diethylacetic acid, methylbutyric acid hydrochloric acid, hydrobromic acid, phosphoric acid, nitric acid, sulfuric acid, butylated hydroxyanisole, butylated hydroxytoluene, monothioglycerol, propyl gallate, sodium ascorbate, sodium bisulfite, sodium metabisulfite, sodium citrate, potassium metabisulfite, potassium sulfite, ammonium acetate, sodium sulfite, tocopherol succinate D-α-tocopheryl polyethylene glycol succinate, D-α-tocopheryl polyethylene glycol 1000 succinate, D-α-tocopherol polyethylene glycol 2000 succinate, and combinations thereof.

7. The nasal dosage form of claim 6, wherein said stabilizers are selected from the group of stabilizers consisting of: citric acid, ascorbic acid, acetic acid, sodium citrate, ammonium acetate, and combinations thereof.

8. The nasal dosage form of claim 6, wherein said stabilizers are selected from the group of stabilizers consisting of: tocopherol succinate D-α-tocopheryl polyethylene glycol succinate, D-α-tocopheryl polyethylene glycol 1000 succinate, D-α-tocopherol polyethylene glycol 2000 succinate or combinations thereof.

9. The nasal dosage form of claim 4, wherein said dosage form comprises dihydroergotamine and stabilizers in a weight ratio of from about 1.0:40.0 to about 40.0:1.0.

10. The nasal dosage form of claim 1, wherein said dosage form does not show any precipitation upon storage at 2° C. to 8° C., 25° C., 40° C., or 45° C. for at least 7 days.

11. The nasal dosage form of claim 1, wherein said dosage form contains total impurities of not more than about 5% when evaluated for at least about 3 months at about 2° C. to about 8° C., or about 25° C. with at least about 60% relative humidity and or about 40° C. with least about 75% relative humidity.

12. A method of administering a pharmaceutical nasal dosage form of dihydroergotamine, comprising: administering the dosage form of dihydroergotamine or a pharmaceutically acceptable salt thereof and at least one pharmaceutically acceptable excipient, wherein said dosage form is administered using a pre-primed nasal device and said dosage form requires less than about 15 minutes and less than four sprays to administer an effective dose of dihydroergotamine for treating migraine with or without aura in human subject.

13. The method of claim 12, wherein said effective dose is from about 0.5 mg to about 2.0 mg of dihydroergotamine.

14. The method of claim 12, wherein said dosage form further comprises one or more stabilizers, wherein said stabiliser are selected from the group consisting of: citric acid, tartaric acid, ascorbic acid, acetic acid, formic acid, methanoic acid, fumaric acid, propionic acid, butanoic acid, ethanoic acid, benzoic acid, butyric acid, malic acid, propionic acid, epoxysuccinic acid, muconic acid, furanacrylic acid, citramalic acid, capric acid, stearic acid, caproic acid, malonic acid, succinic acid, diethylacetic acid, methylbutyric acid hydrochloric acid, hydrobromic acid, phosphoric acid, nitric acid, sulfuric acid, butylated hydroxyanisole, butylated hydroxytoluene, monothioglycerol, propyl gallate, sodium ascorbate, sodium bisulfate, sodium metabisulfite, sodium citrate, potassium metabisulfite, potassium sulfite, ammonium acetate, sodium sulfite, tocopherol succinate D-α-tocopheryl polyethylene glycol succinate, D-α-tocopheryl polyethylene glycol 1000 succinate, D-α-tocopherol polyethylene glycol 2000 succinate, and combinations thereof.

15. The method of claim 12, wherein said dosage from upon intranasal administration to human subjects provides at least about a 10 percent higher dC/dT value compared to a 2 mg dihydroergotamine nasal dosage form and said dC/dT value is measured in a single dose human pharmacokinetic study in a time period of T_{0 min} to T_{15 mins}.

16. The method of claim 15, wherein said dosage form upon intranasal administration to human subjects provides a dC/dT value of at least about 1000 (pg/mL)/hr in a time period of T_{0 min} to T_{15 mins}.

17. The method of claim 12, wherein said dosage form upon intranasal administration to human subjects provides at least about a 10% reduction in coefficient of variance (CV %) of Cₘₐₓ or AUC₍₀₋ₜ₎, AUC_{(0-∞)}, or AUC_{(0-2 hr)}, compared to a 2 mg dihydroergotamine nasal dosage form.

18. The method of claim 12, wherein said dosage form upon intranasal administration to human subjects provides at least about 10 percent higher AUC₍₀₋ₜ₎, AUC_{(0-∞)}, or AUC₍₀₋₂₎, compared to a 2 mg dihydroergotamine nasal dosage form.

19. The method of claim 12, wherein said dosage form upon intranasal administration to human subjects provides at least about 10% reduction in time required to achieve plasma concentration of at least about 700 pg/ml, compared to a 2 mg dihydroergotamine nasal dosage form.

20. The method of claim 12, wherein said dosage form upon intranasal administration to human subjects provides at least one of the following pharmacokinetic parameters:
a. Cₘₐₓ of at least 900 pg/mL;
b. AUC₍₀₋ₜ₎ of at least 4500 pg*hr/mL; and
c. AUC_{(0-∞)} of at least 5000 pg*hr/mL.
